(19) United States
(12) Patent Application Publication
(10) Pub. No.: US 2018/0208970 A1
(43) Pub. Date: Jul. 26, 2018

(54) DIRECT QUANTITATIVE PCR DEVICE AND METHOD OF USE THEREOF

(71) Applicant: America Diagnosis, Inc., San Diego, CA (US)

(72) Inventor: Yong Jiang, San Diego, CA (US)

(21) Appl. No.: 15/877,214

(22) Filed: Jan. 22, 2018

Related U.S. Application Data
(60) Provisional application No. 62/449,322, filed on Jan. 23, 2017.

(51) Int. Cl. C12Q 1/686; B01L 7/00; B01L 3/00

(52) U.S. Cl. CPC C12Q 1/686; B01L 2300/18; B01L 3/502769; B01L 7/52

(57) ABSTRACT

Provided herein are a direct quantitative PCR system, device, and method for analyzing a biological fluid sample.

### CROSS REFERENCE TO RELATED APPLICATION

This application claims the benefit of U.S. Provisional Application No. 62/449,322, filed Jan. 23, 2017; the disclosure of which is incorporated herein by reference in its entirety.

### FIELD

Provided herein are a direct quantitative PCR system, device, and method for analyzing a biological fluid sample.

### BACKGROUND

The polymerase chain reaction (PCR) is a DNA amplification technique, which requires a target DNA, a thermostable DNA polymerase, deoxynucleotide triphosphates (dNTPs), and two specifically designed primers that are complementary to the sequence to be amplified. Direct PCR is a technology that allows PCR amplification directly from a biological sample without DNA extraction and purification.

PCR amplification is carried out in cycles. Each cycle starts with the denaturation of a target double-stranded (ds) DNA by high temperature (e.g., 90-98° C.) to separate its two intertwined strands to form the necessary single-stranded DNA template for replication by a thermostable DNA polymerase (denaturation). By lowering the temperature to approximately 40-60° C., the primers are annealed to the single-stranded DNA template, providing a starting point for the extension of the target DNA by the DNA polymerase (anneraling). The synthesis of new DNA fragments begin as the reaction temperature is raised to the optimum for the DNA polymerase, which is from 70 to 74° C. for most thermostable DNA polymerases (extension). Successive cycles of the denaturation of the target DNA and annealing and extension of the primers produce a large number of copies of a particular DNA segment within a short period of time.

A real-time polymerase chain reaction (RT-PCR), also called quantitative PCR (qPCR), monitors the amplification of a targeted DNA during the PCR process. qPCR can be operated quantitatively and semi-quantitatively, i.e., above/below a certain amount of DNA molecules. qPCR has been used in quantifying nucleic acids, mutation detection, and genotyping analysis. Many different qPCR systems have been developed, including probe-based methods, such as TAQMAN® probes (Heid et al., Genome. Res. 1996, 6, 986-994), molecular beacons (Kostrikis et al., Science 1998, 279, 1228-1229), sunrise primers (Nazarenko et al., Nucleic Acids Res. 1997, 25, 2516-2521), SCORPION® primers (Whitcombe et al., Nat. Biotechnol. 1999, 17, 804-807), and LIGHTUP® probes (Isacsson et al., Mol. Cell. Probes 2000, 14, 321-328). Alternatively, DNA-binding fluorescence dyes, which bind to all dsDNA non-specifically, are used in qPCR to monitor DNA amplification.

PCR is an important tool for medical diagnosis. For example, PCR can be used to detect and identify pathogenic organisms in patients, such as tuberculosis, chlamydia, viral meningitis, viral hepatitis, HIV, and cytomegalovirus. PCR can also be used to diagnose genetic diseases and to identify and characterize genetic mutations and gene rearrangements found in cancers. Therefore, there is a need for an automated diagnostic device for performing a direct quantitative PCR analysis efficiently and reproducibly with minimal human involvement.

### SUMMARY OF THE DISCLOSURE

Provided herein is a direct quantitative PCR system, comprising: a liquid dispenser, a thermal cycler, a light source, and a light detector; wherein the liquid dispenser comprises one or more fluid splitters. In one embodiment, the liquid dispenser further comprises one or more fluid mixers. In another embodiment, the liquid dispenser further comprises a heating unit.

Also provided herein is a direct quantitative PCR system, comprising: a liquid handling module comprising a liquid dispenser, a thermal cycling module, and an imaging module; wherein the liquid dispenser comprises one or more fluid splitters. In one embodiment, the liquid dispenser further comprises one or more fluid mixers. In another embodiment, the liquid dispenser further comprises a heating unit.

Additionally provided herein is a direct quantitative PCR system, comprising: a liquid handling module comprising a liquid dispenser, a thermal cycling module, an imaging module, and a data processing module; wherein the liquid dispenser comprises one or more fluid splitters. In one embodiment, the liquid dispenser further comprises one or more fluid mixers. In another embodiment, the liquid dispenser further comprises a heating unit.

Further provided herein is a direct quantitative PCR system, comprising: a liquid handling module comprising a liquid dispenser, a thermal cycling module, an imaging module, a processor, and a data processing module; wherein the liquid dispenser comprises one or more fluid splitters; and wherein the processor is communicably connected to the liquid dispenser, thermal cycling module, imaging module, and data processing module. In one embodiment, the liquid dispenser further comprises one or more fluid mixers. In another embodiment, the liquid dispenser further comprises a heating unit.

Provided herein is a direct quantitative PCR device, comprising: a liquid dispenser, a thermal cycler, a light source, and a light detector; wherein the liquid dispenser comprises one or more fluid splitters. In one embodiment, the liquid dispenser further comprises one or more fluid mixers. In another embodiment, the liquid dispenser further comprises a heating unit.

Provided herein is a direct quantitative PCR device, comprising: a liquid handling module comprising a liquid dispenser, a thermal cycling module, and an imaging module; wherein the liquid dispenser comprises one or more fluid splitters. In one embodiment, the liquid dispenser further comprises one or more fluid mixers. In another embodiment, the liquid dispenser further comprises a heating unit.

Provided herein is a direct quantitative PCR device, comprising: a liquid handling module comprising a liquid dispenser, a thermal cycling module, an imaging module, and a data processing module; wherein the liquid dispenser comprises one or more fluid splitters. In one embodiment, the liquid dispenser further comprises one or more fluid mixers. In another embodiment, the liquid dispenser further comprises a heating unit.

Provided herein is a direct quantitative PCR device, comprising: a liquid handling module comprising a liquid dispenser, a thermal cycling module, an imaging module, a processor, and a data processing module; wherein the liquid dispenser comprises one or more fluid mixers; and wherein the processor is communicably connected to the liquid dispenser, thermal cycling module, imaging module, and data processing module. In one embodiment, the liquid dispenser further comprises one or more fluid mixers. In another embodiment, the liquid dispenser further comprises a heating unit.

Provided herein is an automated multiple-channel liquid dispenser, comprising a manifold; one or more pumps; one or more dispense heads; one or more fluid mixers; and one or more fluid splitters; wherein the manifold, pumps, dispense heads, fluid mixers, and fluid splitters are in fluid communication.

Provided herein is a method for performing a direct PCR analysis, comprising:

(a) mixing a biological fluid sample to be analyzed with one or more buffer solutions with a liquid dispenser comprising one or more fluid mixers and one or more fluid splitters to form a sample mixture; wherein one of the one or more buffer solutions comprises a fluorescence dye;

(b) dispensing a predetermined volume of the sample mixture into each well of a disposable PCR chip;

(c) determining the fluorescence intensity of the fluorescence dye in each well of the disposable PCR chip;

(d) subjecting the sample mixture in each well of the disposable PCR chip to suitable nucleic acid amplification conditions; and

(e) determining the fluorescence intensity of the fluorescence dye in each well of the disposable PCR chip after amplification.

Provided herein is a method for performing a direct PCR analysis, comprising:

(a) transferring a biological fluid sample to be analyzed and a first buffer solution to a first fluid mixer to form a first sample mixture;

(b) transferring a portion of the first sample mixture using a first fluid splitter and a second buffer solution to a second fluid mixer to form a second sample mixture;

(c) transferring at least a portion of the second sample mixture and a third buffer mixture to a third fluid mixer to form a third sample mixture; wherein the third buffer solution comprises a fluorescence dye;

(d) dispensing a predetermined volume of the third sample mixture into each well of a disposable PCR chip;

(e) determining the fluorescence intensity of the fluorescence dye in each well of the disposable PCR chip;

(f) subjecting the sample mixture in each well of the disposable PCR chip to suitable nucleic acid amplification conditions; and

(g) determining the fluorescence intensity of the fluorescence dye in each well of the disposable PCR chip after amplification.

Provided herein is a method for performing a direct PCR analysis, comprising:

(a) transferring a biological fluid sample to be analyzed and a first buffer solution to a first fluid mixer to form a first sample mixture;

(b) transferring a portion of the first sample mixture using a first fluid splitter and a second buffer solution to a second fluid mixer to form a second sample mixture;

(c) transferring a portion of the second sample mixture using a second fluid splitter and a third buffer mixture to a third fluid mixer to form a third sample mixture; wherein the third buffer solution comprises a fluorescence dye;

(d) dispensing a predetermined volume of the third sample mixture into each well of a disposable PCR chip;

(e) determining the fluorescence intensity of the fluorescence dye in each well of the disposable PCR chip;

(f) subjecting the sample mixture in each well of the disposable PCR chip to suitable nucleic acid amplification conditions; and

(g) determining the fluorescence intensity of the fluorescence dye in each well of the disposable PCR chip after amplification.

Provided herein is an automated method for preparing a biological fluid sample for analysis, which comprises mixing the biological fluid sample to be analyzed with one or more buffer solutions with a liquid dispenser comprising one or more fluid splitters to form a sample mixture. In one embodiment, the liquid dispenser further comprises one or more fluid mixers.

Provided herein is an automated method for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture; and

(c) transferring at least a portion of the second sample mixture and a third buffer mixture to a third fluid mixer to form a third sample mixture.

Provided herein is an automated method for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture; and

(c) transferring a portion of the second sample mixture using a second fluid splitter and a third buffer mixture to a third fluid mixer to form a third sample mixture.

Provided herein is a computer readable medium comprising instructions for performing a direct PCR analysis, comprising:

(a) mixing a biological fluid sample to be analyzed with one or more buffer solutions with a liquid dispenser comprising one or more fluid mixers and one or more fluid splitters to form a sample mixture; wherein one of the one or more buffer solutions comprises a fluorescence dye;

(b) dispensing a predetermined volume of the sample mixture into each well of a disposable PCR chip;

(c) determining the fluorescence intensity of the fluorescence dye in each well of the disposable PCR chip;

(d) subjecting the sample mixture in each well of the disposable PCR chip to suitable nucleic acid amplification conditions; and

(e) determining the fluorescence intensity of the fluorescence dye in each well of the disposable PCR chip after amplification.

Provided herein is a computer readable medium comprising instructions for performing a direct PCR analysis, comprising:

(a) transferring a biological fluid sample to be analyzed and a first buffer solution to a first fluid mixer to form a first sample mixture;

(b) transferring a portion of the first sample mixture using a first fluid splitter and a second buffer solution to a second fluid mixer to form a second sample mixture;

(c) transferring at least a portion of the second sample mixture and a third buffer mixture to a third fluid mixer to form a third sample mixture; wherein the third buffer solution comprises a fluorescence dye;

(d) dispensing a predetermined volume of the third sample mixture into each well of a disposable PCR chip;

(e) determining the fluorescence intensity of the fluorescence dye in each well of the disposable PCR chip;

(f) subjecting the sample mixture in each well of the disposable PCR chip to suitable nucleic acid amplification conditions; and

(g) determining the fluorescence intensity of the fluorescence dye in each well of the disposable PCR chip after amplification.

Provided herein is a computer readable medium comprising instructions for performing a direct PCR analysis, comprising:

(a) transferring a biological fluid sample to be analyzed and a first buffer solution to a first fluid mixer to form a first sample mixture;

(b) transferring a portion of the first sample mixture using a first fluid splitter and a second buffer solution to a second fluid mixer to form a second sample mixture;

(c) transferring a portion of the second sample mixture using a second fluid splitter and a third buffer mixture to a third fluid mixer to form a third sample mixture; wherein the third buffer solution comprises a fluorescence dye;

(d) dispensing a predetermined volume of the third sample mixture into each well of a disposable PCR chip;

(e) determining the fluorescence intensity of the fluorescence dye in each well of the disposable PCR chip;

(f) subjecting the sample mixture in each well of the disposable PCR chip to suitable nucleic acid amplification conditions; and

(g) determining the fluorescence intensity of the fluorescence dye in each well of the disposable PCR chip after amplification.

Provided herein is a computer readable medium comprising instructions for preparing a biological fluid sample for analysis, which comprises mixing the biological fluid sample to be analyzed with one or more buffer solutions with a liquid dispenser comprising one or more fluid splitters to form a sample mixture. In one embodiment, the liquid dispenser further comprises one or more fluid mixers.

Provided herein is a computer readable medium comprising instructions for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture; and

(c) transferring at least a portion of the second sample mixture and a third buffer mixture to a third fluid mixer to form a third sample mixture.

Provided herein is a computer readable medium comprising instructions for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture; and

(c) transferring a portion of the second sample mixture using a second fluid splitter and a third buffer mixture to a third fluid mixer to form a third sample mixture.

Provided herein is a method for performing a direct PCR analysis, comprising:

(a) transferring a biological fluid sample to be analyzed and a first buffer solution to a first fluid mixer to form a first sample mixture;

(b) heating the first sample mixture at a temperature ranging from about 30 to about 100° C. for a period ranging from about 30 seconds to about 30 minutes;

(c) transferring a portion of the first sample mixture using a first fluid splitter and a second buffer solution to a second fluid mixer to form a second sample mixture;

(d) transferring at least a portion of the second sample mixture and a third buffer mixture to a third fluid mixer to form a third sample mixture; wherein the third buffer solution comprises a fluorescence dye;

(e) dispensing a predetermined volume of the third sample mixture into each well of a disposable PCR chip;

(f) determining the fluorescence intensity of the fluorescence dye in each well of the disposable PCR chip;

(g) subjecting the sample mixture in each well of the disposable PCR chip to suitable nucleic acid amplification conditions; and

(h) determining the fluorescence intensity of the fluorescence dye in each well of the disposable PCR chip after amplification.

Provided herein is a method for performing a direct PCR analysis, comprising:

(a) transferring a biological fluid sample to be analyzed and a first buffer solution to a first fluid mixer to form a first sample mixture;

(b) transferring a portion of the first sample mixture using a first fluid splitter and a second buffer solution to a second fluid mixer to form a second sample mixture;

(c) heating the second sample mixture at a temperature ranging from about 30 to about 100° C. for a period ranging from about 30 seconds to about 30 minutes;

(d) transferring at least a portion of the second sample mixture and a third buffer mixture to a third fluid mixer to form a third sample mixture; wherein the third buffer solution comprises a fluorescence dye;

(e) dispensing a predetermined volume of the third sample mixture into each well of a disposable PCR chip;

(f) determining the fluorescence intensity of the fluorescence dye in each well of the disposable PCR chip;

(g) subjecting the sample mixture in each well of the disposable PCR chip to suitable nucleic acid amplification conditions; and

(h) determining the fluorescence intensity of the fluorescence dye in each well of the disposable PCR chip after amplification.

Provided herein is a method for performing a direct PCR analysis, comprising:

(a) transferring a biological fluid sample to be analyzed and a first buffer solution to a first fluid mixer to form a first sample mixture;

(b) heating the first sample mixture at a temperature ranging from about 30 to about 100° C. for a period ranging from about 30 seconds to about 30 minutes;

(c) transferring a portion of the first sample mixture using a first fluid splitter and a second buffer solution to a second fluid mixer to form a second sample mixture;

(d) transferring a portion of the second sample mixture using a second fluid splitter and a third buffer mixture to a third fluid mixer to form a third sample mixture; wherein the third buffer solution comprises a fluorescence dye;

(e) dispensing a predetermined volume of the third sample mixture into each well of a disposable PCR chip;

(f) determining the fluorescence intensity of the fluorescence dye in each well of the disposable PCR chip;

(g) subjecting the sample mixture in each well of the disposable PCR chip to suitable nucleic acid amplification conditions; and

(h) determining the fluorescence intensity of the fluorescence dye in each well of the disposable PCR chip after amplification.

Provided herein is a method for performing a direct PCR analysis, comprising:

(a) transferring a biological fluid sample to be analyzed and a first buffer solution to a first fluid mixer to form a first sample mixture;

(b) transferring a portion of the first sample mixture using a first fluid splitter and a second buffer solution to a second fluid mixer to form a second sample mixture;

(c) heating the second sample mixture at a temperature ranging from about 30 to about 100° C. for a period ranging from about 30 seconds to about 30 minutes;

(d) transferring a portion of the second sample mixture using a second fluid splitter and a third buffer mixture to a third fluid mixer to form a third sample mixture; wherein the third buffer solution comprises a fluorescence dye;

(e) dispensing a predetermined volume of the third sample mixture into each well of a disposable PCR chip;

(f) determining the fluorescence intensity of the fluorescence dye in each well of the disposable PCR chip;

(g) subjecting the sample mixture in each well of the disposable PCR chip to suitable nucleic acid amplification conditions; and

(h) determining the fluorescence intensity of the fluorescence dye in each well of the disposable PCR chip after amplification.

Provided herein is an automated method for preparing a biological fluid sample for analysis, comprising:

(a) transferring the biological fluid sample and a first buffer solution to a first fluid mixer to form a first sample mixture;

(b) heating the first sample mixture at a temperature ranging from about 30 to about 100° C. for a period ranging from 30 seconds to about 30 minutes;

(c) transferring a portion of the first sample mixture using a first fluid splitter and a second buffer solution to a second fluid mixer to form a second sample mixture; and

(d) transferring at least a portion of the second sample mixture and a third buffer mixture to a third fluid mixer to form a third sample mixture.

Provided herein is an automated method for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture;

(c) heating the second sample mixture at a temperature ranging from about 30 to about 100° C. for a period ranging from about 30 seconds to about 30 minutes; and

(d) transferring at least a portion of the second sample mixture and a third buffer mixture to a third fluid mixer to form a third sample mixture.

Provided herein is an automated method for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture;

(c) transferring at least a portion of the second sample mixture and a third buffer mixture to a third fluid mixer to form a third sample mixture; and

(d) heating the third sample mixture at a temperature ranging from about 30 to about 100° C. for a period ranging from about 30 seconds to about 30 minutes.

Provided herein is an automated method for preparing a biological fluid sample for analysis, comprising:

(a) transferring the biological fluid sample and a first buffer solution to a first fluid mixer to form a first sample mixture;

(b) heating the first sample mixture at a temperature ranging from about 30 to about 100° C. for a period ranging from about 30 seconds to about 30 minutes;

(c) transferring a portion of the first sample mixture using a first fluid splitter and a second buffer solution to a second fluid mixer to form a second sample mixture; and

(d) transferring a portion of the second sample mixture using a second fluid splitter and a third buffer mixture to a third fluid mixer to form a third sample mixture.

Provided herein is an automated method for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture;

(c) heating the second sample mixture at a temperature ranging from about 30 to about 100° C. for a period ranging from about 30 seconds to about 30 minutes; and

(d) transferring a portion of the second sample mixture using a second fluid splitter and a third buffer mixture to a third fluid mixer to form a third sample mixture.

Provided herein is an automated method for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture;

(c) transferring a portion of the second sample mixture using a second fluid splitter and a third buffer mixture to a third fluid mixer to form a third sample mixture; and

(d) heating the third sample mixture at a temperature ranging from about 30 to about 100° C. for a period ranging from about 30 seconds to about 30 minutes.

Provided herein is a computer readable medium comprising instructions for performing a direct PCR analysis, comprising:

(a) transferring a biological fluid sample to be analyzed and a first buffer solution to a first fluid mixer to form a first sample mixture;

(b) heating the first sample mixture at a temperature ranging from about 30 to about 100° C. for a period ranging from about 30 seconds to about 30 minutes;

(c) transferring a portion of the first sample mixture using a first fluid splitter and a second buffer solution to a second fluid mixer to form a second sample mixture;

(d) transferring at least a portion of the second sample mixture and a third buffer mixture to a third fluid mixer to form a third sample mixture; wherein the third buffer solution comprises a fluorescence dye;

(e) dispensing a predetermined volume of the third sample mixture into each well of a disposable PCR chip;

(f) determining the fluorescence intensity of the fluorescence dye in each well of the disposable PCR chip;

(g) subjecting the sample mixture in each well of the disposable PCR chip to suitable nucleic acid amplification conditions; and

(h) determining the fluorescence intensity of the fluorescence dye in each well of the disposable PCR chip after amplification.

Provided herein is a computer readable medium comprising instructions for performing a direct PCR analysis, comprising:

(a) transferring a biological fluid sample to be analyzed and a first buffer solution to a first fluid mixer to form a first sample mixture;

(b) transferring a portion of the first sample mixture using a first fluid splitter and a second buffer solution to a second fluid mixer to form a second sample mixture;

(c) heating the second sample mixture at a temperature ranging from about 30 to about 100° C. for a period ranging from about 30 seconds to about 30 minutes;

(d) transferring at least a portion of the second sample mixture and a third buffer mixture to a third fluid mixer to form a third sample mixture; wherein the third buffer solution comprises a fluorescence dye;

(e) dispensing a predetermined volume of the third sample mixture into each well of a disposable PCR chip;

(f) determining the fluorescence intensity of the fluorescence dye in each well of the disposable PCR chip;

(g) subjecting the sample mixture in each well of the disposable PCR chip to suitable nucleic acid amplification conditions; and

(h) determining the fluorescence intensity of the fluorescence dye in each well of the disposable PCR chip after amplification.

Provided herein is a computer readable medium comprising instructions for performing a direct PCR analysis, comprising:

(a) transferring a biological fluid sample to be analyzed and a first buffer solution to a first fluid mixer to form a first sample mixture;

(b) heating the first sample mixture at a temperature ranging from about 30 to about 100° C. for a period ranging from about 30 seconds to about 30 minutes;

(c) transferring a portion of the first sample mixture using a first fluid splitter and a second buffer solution to a second fluid mixer to form a second sample mixture;

(d) transferring a portion of the second sample mixture using a second fluid splitter and a third buffer mixture to a third fluid mixer to form a third sample mixture; wherein the third buffer solution comprises a fluorescence dye;

(e) dispensing a predetermined volume of the third sample mixture into each well of a disposable PCR chip;

(f) determining the fluorescence intensity of the fluorescence dye in each well of the disposable PCR chip; (g) subjecting the sample mixture in each well of the disposable PCR chip to suitable nucleic acid amplification conditions; and

(h) determining the fluorescence intensity of the fluorescence dye in each well of the disposable PCR chip after amplification.

Provided herein is a computer readable medium comprising instructions for performing a direct PCR analysis, comprising:

(a) transferring a biological fluid sample to be analyzed and a first buffer solution to a first fluid mixer to form a first sample mixture;

(b) transferring a portion of the first sample mixture using a first fluid splitter and a second buffer solution to a second fluid mixer to form a second sample mixture;

(c) heating the second sample mixture at a temperature ranging from about 30 to about 100° C. for a period ranging from about 30 seconds to about 30 minutes;

(d) transferring a portion of the second sample mixture using a second fluid splitter and a third buffer mixture to a third fluid mixer to form a third sample mixture; wherein the third buffer solution comprises a fluorescence dye;

(e) dispensing a predetermined volume of the third sample mixture into each well of a disposable PCR chip;

(f) determining the fluorescence intensity of the fluorescence dye in each well of the disposable PCR chip;

(g) subjecting the sample mixture in each well of the disposable PCR chip to suitable nucleic acid amplification conditions; and

(h) determining the fluorescence intensity of the fluorescence dye in each well of the disposable PCR chip after amplification.

Provided herein is a computer readable medium comprising instructions for preparing a biological fluid sample for analysis, comprising:

(a) transferring the biological fluid sample and a first buffer solution to a first fluid mixer to form a first sample mixture;

(b) heating the first sample mixture at a temperature ranging from about 30 to about 100° C. for a period ranging from about 30 seconds to about 30 minutes;

(c) transferring a portion of the first sample mixture using a first fluid splitter and a second buffer solution to a second fluid mixer to form a second sample mixture; and

(d) transferring at least a portion of the second sample mixture and a third buffer mixture to a third fluid mixer to form a third sample mixture.

Provided herein is a computer readable medium comprising instructions for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture;

(c) heating the second sample mixture at a temperature ranging from about 30 to about 100° C. for a period ranging from about 30 seconds to about 30 minutes; and

(d) transferring at least a portion of the second sample mixture and a third buffer mixture to a third fluid mixer to form a third sample mixture.

Provided herein is a computer readable medium comprising instructions for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture;

(c) transferring at least a portion of the second sample mixture and a third buffer mixture to a third fluid mixer to form a third sample mixture; and

(d) heating the third sample mixture at a temperature ranging from about 30 to about 100° C. for a period ranging from about 30 seconds to about 30 minutes.

Provided herein is a computer readable medium comprising instructions for preparing a biological fluid sample for analysis, comprising:

(a) transferring the biological fluid sample and a first buffer solution to a first fluid mixer to form a first sample mixture;

(b) heating the first sample mixture at a temperature ranging from about 30 to about 100° C. for a period ranging from about 30 seconds to about 30 minutes;

(c) transferring a portion of the first sample mixture using a first fluid splitter and a second buffer solution to a second fluid mixer to form a second sample mixture; and

(d) transferring a portion of the second sample mixture using a second fluid splitter and a third buffer mixture to a third fluid mixer to form a third sample mixture.

Provided herein is a computer readable medium comprising instructions for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture;

(c) heating the second sample mixture at a temperature ranging from about 30 to about 100° C. for a period ranging from about 30 seconds to about 30 minutes; and

(d) transferring a portion of the second sample mixture using a second fluid splitter and a third buffer mixture to a third fluid mixer to form a third sample mixture.

Provided herein is a computer readable medium comprising instructions for preparing a biological fluid sample for analysis, comprising:

(a) transferring the biological fluid sample and a first buffer solution to a first fluid mixer to form a first sample mixture;

(b) transferring a portion of the first sample mixture using a first fluid splitter and a second buffer solution to a second fluid mixer to form a second sample mixture;

(c) transferring a portion of the second sample mixture using a second fluid splitter and a third buffer mixture to a third fluid mixer to form a third sample mixture; and

(d) heating the third sample mixture at a temperature ranging from about 30 to about 100° C. for a period ranging from about 30 seconds to about 30 minutes.

### BRIEF DESCRIPTION OF THE DRAWINGS

FIG. 1 is a schematic representation of an embodiment of an automated direct quantitative PCR system 1 for analyzing a biological fluid sample without DNA extraction and purification.

FIG. 2 is a schematic representation of an embodiment of an automated direct quantitative PCR system 1 for analyzing a biological fluid sample without DNA extraction and purification, wherein a liquid handling module 11 comprises a liquid dispenser 111, a sample reader 112, and one or more reservoirs 30; and wherein the one or more reservoirs 30 are fluidly connected to the liquid dispenser 111.

FIG. 3 is a schematic representation of an embodiment of an automated direct quantitative PCR system 1 for analyzing a biological fluid sample without DNA extraction and purification, wherein a thermal cycling module 12 comprises a thermal cycler 121, a chip reader 122, and one or more disposable PCR chips 123.

FIG. 4 is a schematic representation of an embodiment of an automated direct quantitative PCR system 1 for analyzing a biological fluid sample without DNA extraction and purification, wherein a thermal cycling module 12 comprises a thermal cycler 121, a chip reader 122, one or more disposable PCR chips 123, a robotic arm 124, and a chip storage unit 125.

FIG. 5 is a schematic representation of an embodiment of an automated direct quantitative PCR system 1 for analyzing a biological fluid sample without DNA extraction and purification, wherein an imaging module 13 comprises a light source 131 and a light detector 132.

FIG. 6 is a schematic representation of an embodiment of an automated direct quantitative PCR device 2 for analyzing a biological fluid sample without DNA extraction and purification; wherein a liquid handling module 11 comprises a liquid dispenser 111 and a sample reader 112; a thermal cycling module 12 comprises a thermal cycler 121, a chip reader 122, and one or more disposable PCR chips 123; and an imaging module 13 comprises a light source 131 and a light detector 132.

FIG. 7 is a schematic representation of an embodiment of an automated direct quantitative PCR device 2 for analyzing a biological fluid sample without DNA extraction and purification; wherein a liquid handling module 11 comprises a liquid dispenser 111 and a sample reader 112; a thermal cycling module 12 comprises a thermal cycler 121, a chip reader 122, one or more disposable PCR chips 123, a robotic arm 124, and a chip storage unit 125; and an imaging module 13 comprises a light source 131 and a light detector 132.

FIG. 8 is a schematic representation of an embodiment of an automated direct quantitative PCR device 2 for analyzing a biological fluid sample without DNA extraction and purification; wherein a liquid handling module 11 comprises a liquid dispenser 111 and a sample reader 112; a thermal cycling module 12 comprises a thermal cycler 121, a chip reader 122, and one or more disposable PCR chips 123; and an imaging module 13 comprises a light source 131 and a light detector 132; and wherein the automated direct quantitative PCR device 2 is configured to be in communication with a control device 3 that comprises a processor 21, a data processing module 22, and a user interface 23.

FIG. 9 is a schematic representation of an embodiment of an automated direct quantitative PCR device 2 for analyzing a biological fluid sample without DNA extraction and purification; wherein a liquid handling module 11 comprises a liquid dispenser 111 and a sample reader 112; a thermal cycling module 12 comprises a thermal cycler 121, a chip reader 122, one or more disposable PCR chips 123, a robotic arm 124, and a chip storage unit 125; and an imaging module 13 comprises a light source 131 and a light detector 132; and wherein the automated direct quantitative PCR device 2 is configured to be in communication with a control device 3 that comprises a processor 21, a data processing module 22, and a user interface 23.

FIG. 10 is a schematic representation of an embodiment of an automated direct quantitative PCR device 2 for analyzing a biological fluid sample without DNA extraction and purification, wherein a liquid dispenser 111 aspirates a biological fluid sample from a sample container 4 to dispense the sample via nozzles 1111 into wells 1231 of a disposable PCR chip 123 after the sample is mixed with a lysis reagent, a neutralizing reagent, and a PCR reaction solution.

FIG. 11 is a top view of an embodiment of a disposable PCR chip 123 comprising 8×8 wells 1231 and a barcode 1232.

FIG. 12 is a perspective view of an embodiment of a thermal cycler module 12, illustrating the engagement of a robotic arm 124 with a disposable PCR chip 123 for transfer.

FIG. 13 is a perspective view of an embodiment of a thermal cycler module 12, illustrating a robotic arm 124 retrieving a disposable PCR chip 123 from a chip storage unit 125.

FIG. 14 is a cross-sectional view of an embodiment of a thermal cycler module 12, illustrating the engagement of a thermal cycler 121 with a disposable PCR chip 123 for efficient thermal transfer.

FIG. 15A is a schematic representation of an embodiment of an imaging module 13, illustrating the arrangement of a light source 131 and light detector 132 in relation to a thermal cycler 121 and a disposable PCR chip 123.

FIG. 15B is a schematic representation of an embodiment of an imaging module 13, illustrating the arrangement of two light sources 131 and light detector 132 in relation to a thermal cycler 121 and a disposable PCR chip 123, wherein the detection is performed from the top face of the disposable PCR chip 123.

FIG. 15C is a schematic representation of an embodiment of an imaging module 13, illustrating the arrangement of a light source 131 and light detector 132 in relation to a disposable PCR chip 123, wherein the detection is performed from the bottom face of the disposable PCR chip 123.

FIG. 15D is a schematic representation of an embodiment of an imaging module 13, illustrating the arrangement of two light sources 131 and light detector 132 in relation to a disposable PCR chip 123, wherein the detection is performed from the bottom face of the disposable PCR chip 123.

FIG. 16 is a top view of an embodiment of a thermal cycler module 12, illustrating the arrangement of four disposable PCR chips 123 on the top of a translationally movable thermal cycler 121.

FIG. 17 is a top view of an embodiment of a thermal cycler module 12, illustrating the arrangement of four disposable PCR chips 123 on the top of a rotatable thermal cycler 121.

FIG. 18 is a schematic representation of an embodiment of an automated liquid dispenser 111 comprising three fluid mixers (1111, 1113, and 1115), a fluid splitter 1112, and dispenser head 1116; wherein the fluid mixer 1111 is in fluid communication with the reservoir 301 containing a first solution (e.g., a neutralizing buffer solution), the fluid mixer 1113 is in fluid communication with the reservoir 302 containing a second first solution (e.g., a dilution buffer solution such as a phosphate buffered saline (PBS) solution), the fluid mixer 1115 is in fluid communication with the reservoir 303 containing a third solution (e.g., a PCR mixture comprising a fluorescence dye), and the fluid splitter 1112 is in fluid communication with a waste reservoir 401; and wherein the fluid mixers (1111, 1113, and 1115), fluid splitter 1112, and the dispenser head 1116 are in fluid communication.

FIG. 19 is a schematic representation of an embodiment of an automated liquid dispenser 111 comprising three fluid mixers (1111, 1113, and 1115), two fluid splitters (1112 and 1114), and dispenser head 1116; wherein the fluid mixer 1111 is in fluid communication with the reservoir 301 containing a first solution (e.g., a neutralizing buffer solution), the fluid mixer 1113 is in fluid communication with the reservoir 302 containing a second first solution (e.g., a dilution buffer solution such as a phosphate buffered saline (PBS) solution), the fluid mixer 1115 is in fluid communication with the reservoir 303 containing a third solution (e.g., a PCR mixture comprising a fluorescence dye), the fluid splitters 1112 and 1114 are each in fluid communication with a waste reservoir 401; and wherein the fluid mixers (1111, 1113, and 1115), fluid splitters (1112 and 1114), and the dispenser head 1116 are in fluid communication.

FIG. 20 shows a SNP analysis of the CYP2D6 gene.

### DETAILED DESCRIPTION

To facilitate understanding of the disclosure set forth herein, a number of terms are defined below.

Generally, the nomenclature used herein is those well known and commonly employed in the art. Unless defined otherwise, all technical and scientific terms used herein generally have the same meaning as commonly understood by one of ordinary skill in the art to which this disclosure belongs.

The term “module” means an assembly of components, each of which may have separate, distinct and/or independent functions, but which are configured to operate together to produce a desired result or results. It is not required that every component within a module be directly connected or in direct communication with every other. Furthermore, connectivity amongst the various components may be achieved with the aid of a component, such as a processor, that is external to the module.

The term “well” means a discrete concave feature in a material having a surface opening (aperture) that is completely surrounded by interstitial region(s) of the surface. A well can have characteristics such as size (e.g., volume, diameter, and depth), cross-sectional shape (e.g., round, elliptical, triangular, square, polygonal, star shaped (having any suitable number of vertices), irregular, or having concentric wells separated by a dielectric material), and distribution (e.g., spatial locations of the wells within the dielectric material, e.g., regularly spaced or periodic locations, or irregularly spaced or aperiodic locations). The cross section of a well can be, but need not necessarily be, uniform along the length of the well.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Direct Quantitative PCR System

In one embodiment, provided herein is an automated direct quantitative PCR system 1, comprising a liquid handling module 11, a thermal cycling module 12, and an imaging module 13. In one embodiment, the liquid handling module 11 comprises a liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In one embodiment, the automated direct quantitative PCR system 1 provided herein further comprises a processor 21; wherein the processor 21 is communicably connected to the liquid handling module 11, thermal cycling module 12, and imaging module 13. In another embodiment, the automated direct quantitative PCR system provided herein further comprises a processor 21, and a data processing module 22 and/or a user interface 23; wherein the processor 21 is communicably connected to the liquid handling module 11, thermal cycling module 12, imaging module 13, data processing module 22, and user interface 23.

In another embodiment, provided herein is an automated direct quantitative PCR system 1, comprising a liquid handling module 11 comprising a liquid dispenser 111; a thermal cycling module 12, and an imaging module 13; wherein the liquid dispenser 111 comprises, in one embodiment, one or more fluid mixers 1111, in another embodiment, one or more fluid splitters 1112; and in yet another embodiment, one or more fluid mixers 1111 and one or more fluid splitters 1112. In certain embodiments, the liquid dispenser 111 further comprises a heating unit 1120.

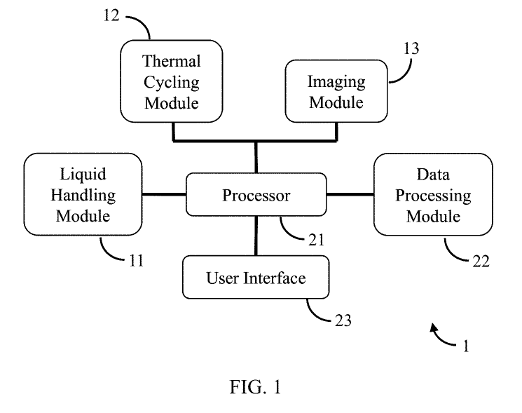

In one embodiment, as illustrated in FIG. 1, the automated direct quantitative PCR system 1 provided herein comprises a liquid handling module 11, a thermal cycling module 12, an imaging module 13, a processor 21, a data processing module 22, and a user interface 23; wherein the processor 21 is communicably connected to the liquid handling module 11, thermal cycling module 12, imaging module 13, data processing module 22, and user interface 23. In one embodiment, the liquid handling module 11 comprises a liquid dispenser 111, wherein the processor 21 is communicably connected to the liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In another embodiment, the automated direct quantitative PCR system 1 provided herein comprises a liquid handling module 11 comprising a liquid dispenser 111, a thermal cycling module 12, an imaging module 13, a processor 21, a data processing module 22, a user interface 23, and one or more reservoirs 30; wherein the processor 21 is communicably connected to the liquid dispenser 111, thermal cycling module 12, imaging module 13, data processing module 22, and user interface 23; and wherein the liquid dispenser 111 is fluidly connected to the reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

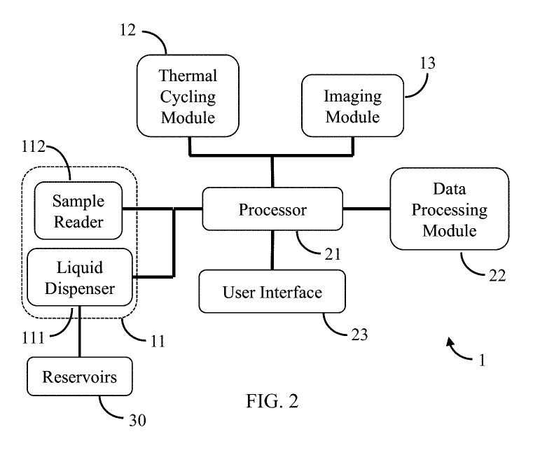

In yet another embodiment, as illustrated in FIG. 2, the automated direct quantitative PCR system 1 provided herein comprises a liquid handling module 11 comprising a liquid dispenser 111 and a sample reader 112, a thermal cycling module 12, an imaging module 13, a processor 21, a data processing module 22, a user interface 23, and one or more reservoirs 30; wherein the processor 21 is communicably connected to the liquid dispenser 111, sample reader 112, thermal cycling module 12, imaging module 13, data processing module 22, and user interface 23; and wherein the liquid dispenser 111 is fluidly connected to the reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

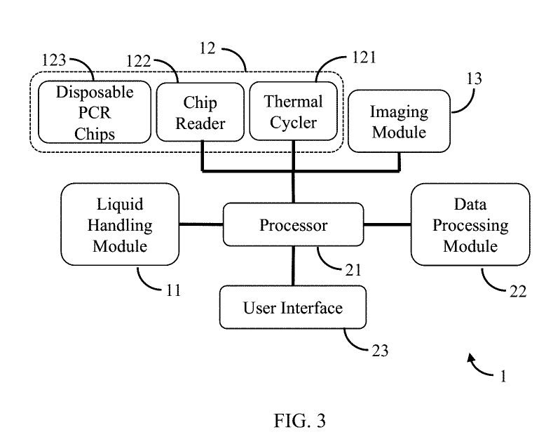

In yet another embodiment, as illustrated in FIG. 3, the automated direct quantitative PCR system 1 provided herein comprises a liquid handling module 11; a thermal cycling module 12 comprising a thermal cycler 121, a chip reader 122, and one or more disposable PCR chips 123; an imaging module 13; a processor 21; a data processing module 22; and a user interface 23; wherein the processor 21 is communicably connected to the liquid handling module 11, thermal cycler 121, chip reader 122, imaging module 13, data processing module 22, and user interface 23. In one embodiment, the liquid handling module 11 comprises a liquid dispenser 111, wherein the processor 21 is communicably connected to the liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

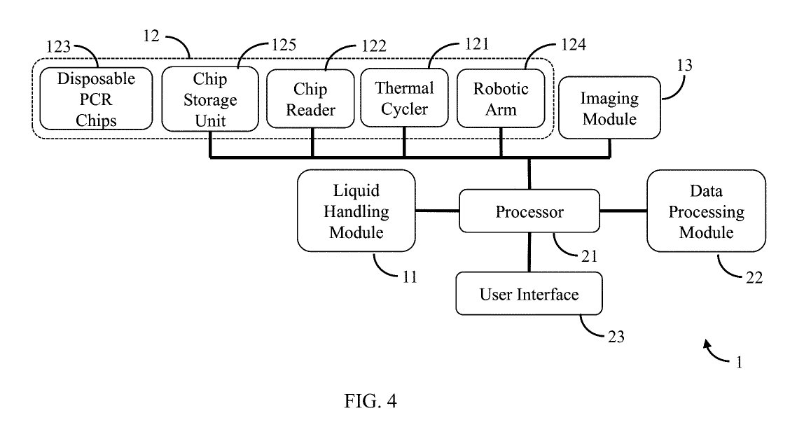

In yet another embodiment, as illustrated in FIG. 4, the automated direct quantitative PCR system 1 provided herein comprises a liquid handling module 11; a thermal cycling module 12 comprising a thermal cycler 121, a chip reader 122, one or more disposable PCR chips 123, a robotic arm 124, and a chip storage unit 125; an imaging module 13; a processor 21; a data processing module 22; and a user interface 23; wherein the processor 21 is communicably connected to the liquid handling module 11, thermal cycler 121, chip reader 122, robotic arm 124, chip storage unit 125, imaging module 13, data processing module 22, and user interface 23. In one embodiment, the liquid handling module 11 comprises a liquid dispenser 111, wherein the processor 21 is communicably connected to the liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

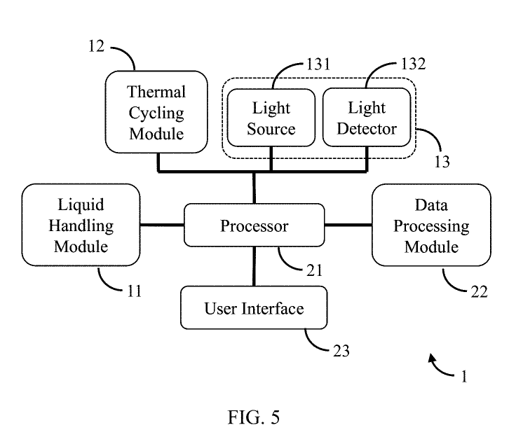

In yet another embodiment, as illustrated in FIG. 5, the automated direct quantitative PCR system 1 provided herein comprises a liquid handling module 11, a thermal cycling module 12, an imaging module 13 comprising a light source 131 and a light detector 132, a processor 21, a data processing module 22, and a user interface 23; wherein the processor 21 is communicably connected to the liquid handling module 11, thermal cycling module 12, light source 131, light detector 132, data processing module 22, and user interface 23. In one embodiment, the liquid handling module 11 comprises a liquid dispenser 111, wherein the processor 21 is communicably connected to the liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR system 1 provided herein comprises a liquid handling module 11 comprising a liquid dispenser 111; a thermal cycling module 12 comprising a thermal cycler 121, a chip reader 122, and one or more disposable PCR chips 123; an imaging module 13 comprising a light source 131 and a light detector 132; a processor 21; a data processing module 22; a user interface 23; and one or more reservoirs 30; wherein the processor 21 is communicably connected to the liquid dispenser 111, thermal cycler 121, chip reader 122, light source 131, light detector 132, data processing module 22, and user interface 23; and wherein the liquid dispenser 111 is fluidly connected to the reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

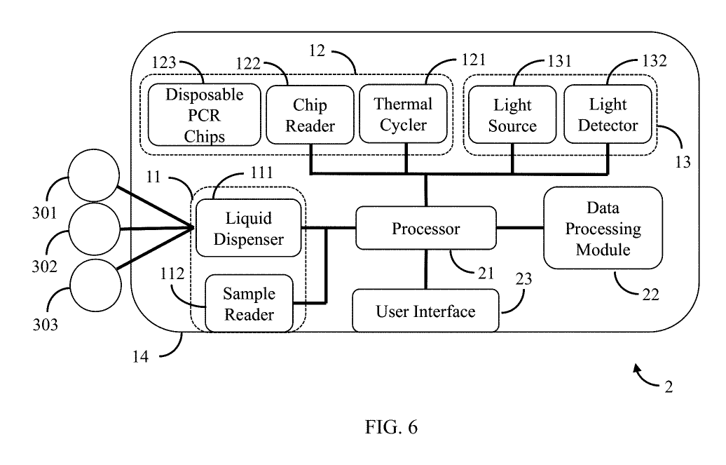

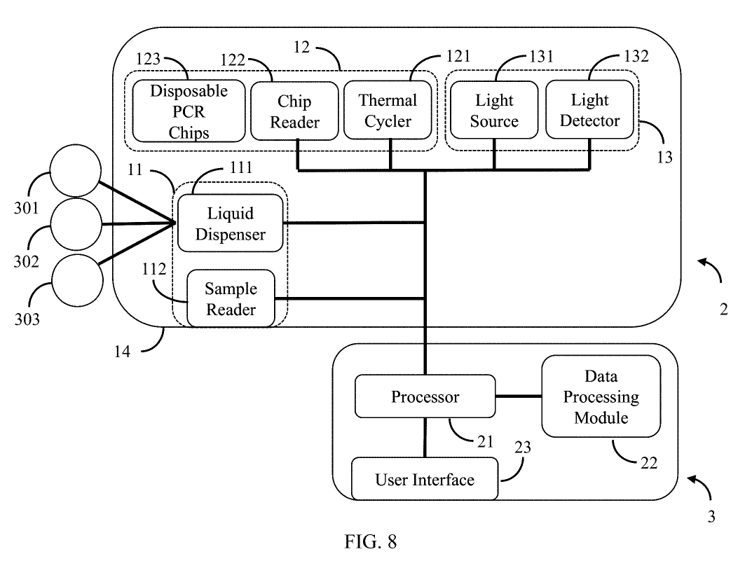

In yet another embodiment, as illustrated in FIG. 6 or 8, the automated direct quantitative PCR system 1 provided herein comprises a liquid handling module 11 comprising a liquid dispenser 111 and a sample reader 112; a thermal cycling module 12 comprising a thermal cycler 121, a chip reader 122, and one or more disposable PCR chips 123; an imaging module 13 comprising a light source 131 and a light detector 132; a processor 21; a data processing module 22; a user interface 23; and reservoirs 301, 302, and 303; wherein the processor 21 is communicably connected to the liquid dispenser 111, sample reader 112, thermal cycler 121, chip reader 122, light source 131, light detector 132, data processing module 22, and user interface 23; and wherein the liquid dispenser 111 is fluidly connected to the reservoirs 301, 302, and 303. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR system 1 provided herein comprises a liquid handling module 11 comprising a liquid dispenser 111; a thermal cycling module 12 comprising a thermal cycler 121, a chip reader 122, and one or more disposable PCR chips 123; an imaging module 13 comprising a light source 131 and a light detector 132; a control device 3; and one or more reservoirs 30; wherein the control device 3 is communicably connected to the liquid dispenser 111, thermal cycler 121, chip reader 122, light source 131, and light detector 132; and wherein the liquid dispenser 111 is fluidly connected to the reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, as illustrated in FIG. 8, the automated direct quantitative PCR system 1 provided herein comprises a liquid handling module 11 comprising a liquid dispenser 111 and a sample reader 112; a thermal cycling module 12 comprising a thermal cycler 121, a chip reader 122, and one or more disposable PCR chips 123; an imaging module 13 comprising a light source 131 and a light detector 132; a control device 3; and reservoirs 301, 302, and 303; wherein the control device 3 is communicably connected to the liquid dispenser 111, sample reader 112, thermal cycler 121, chip reader 122, light source 131, and light detector 132; and wherein the liquid dispenser 111 is fluidly connected to the reservoirs 301, 302, and 303. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR system 1 provided herein comprises a liquid handling module 11 comprising a liquid dispenser 111; a thermal cycling module 12 comprising a thermal cycler 121, a chip reader 122, one or more disposable PCR chips 123, a robotic arm 124, and a chip storage unit 125; an imaging module 13 comprising a light source 131 and a light detector 132; a processor 21; a data processing module 22; a user interface 23; and one or more reservoirs 30; wherein the processor 21 is communicably connected to the liquid dispenser 111, thermal cycler 121, chip reader 122, robotic arm 124, chip storage unit 125, light source 131, light detector 132, data processing module 22, and user interface 23; and wherein the liquid dispenser 111 is fluidly connected to the reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

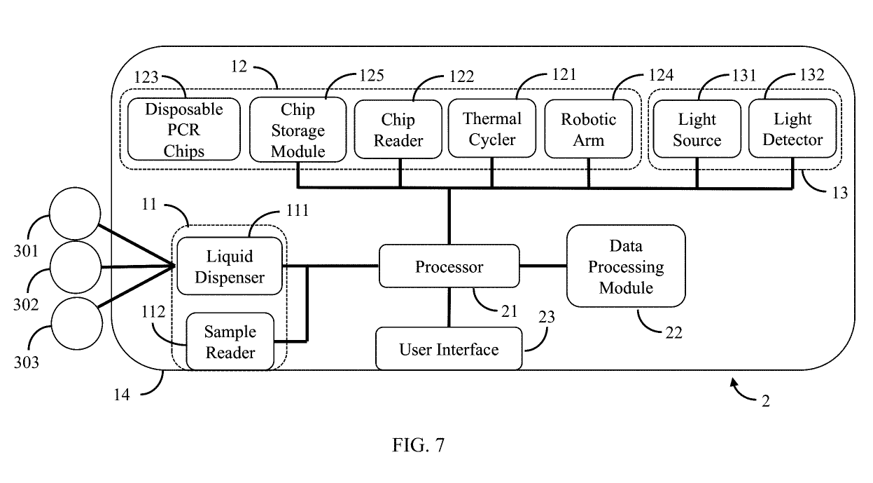

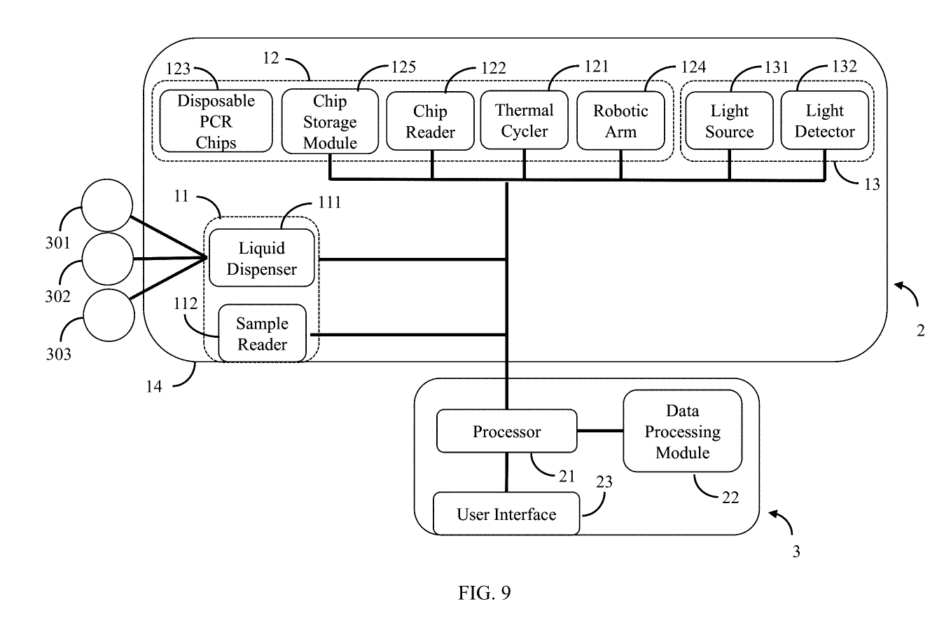

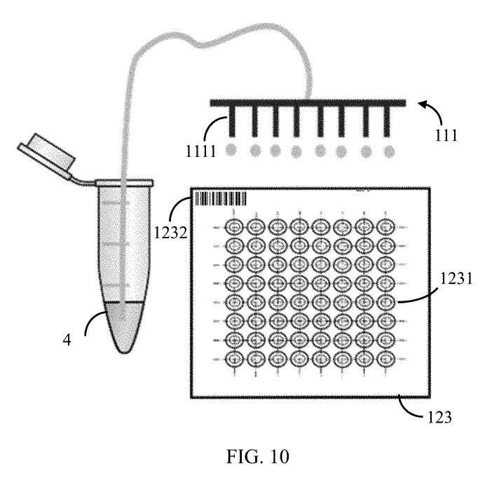

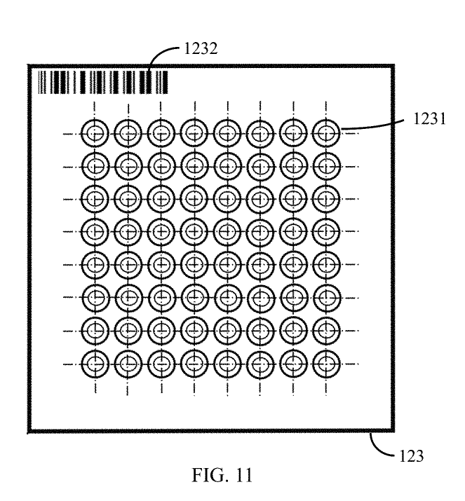

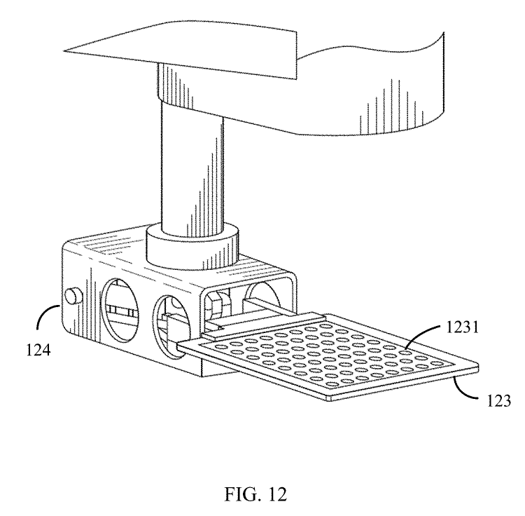

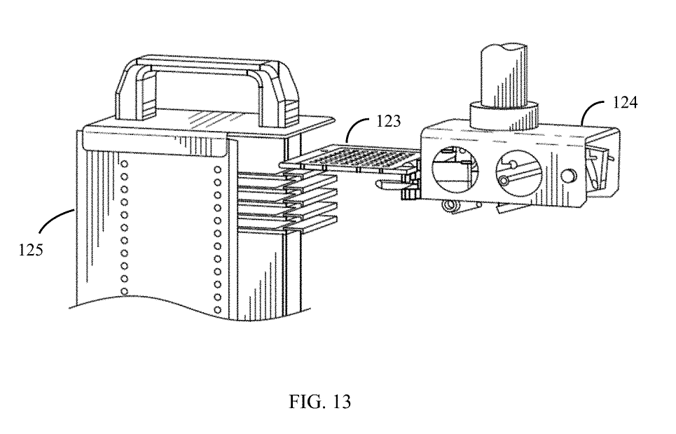

In yet another embodiment, as illustrated in FIG. 7 or 9, the automated direct quantitative PCR system 1 provided herein comprises a liquid handling module 11 comprising a liquid dispenser 111 and a sample reader 112; a thermal cycling module 12 comprising a thermal cycler 121, a chip reader 122, one or more disposable PCR chips 123, a robotic arm 124, and a chip storage unit 125; an imaging module 13 comprising a light source 131 and a light detector 132; a processor 21; a data processing module 22; a user interface 23; and reservoirs 301, 302, and 303; wherein the processor 21 is communicably connected to the liquid dispenser 111, sample reader 112, thermal cycler 121, chip reader 122, robotic arm 124, chip storage unit 125, light source 131, light detector 132, data processing module 22, and user interface 23; and wherein the liquid dispenser 111 is fluidly connected to the reservoirs 301, 302, and 303. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR system 1 provided herein comprises a liquid handling module 11 comprising a liquid dispenser 111; a thermal cycling module 12 comprising a thermal cycler 121, a chip reader 122, one or more disposable PCR chips 123, a robotic arm 124, and a chip storage unit 125; an imaging module 13 comprising a light source 131 and a light detector 132; a control device 3; and one or more reservoirs 30; wherein the control device 3 is communicably connected to the liquid dispenser 111, thermal cycler 121, chip reader 122, robotic arm 124, chip storage unit 125, light source 131, and light detector 132; and wherein the liquid dispenser 111 is fluidly connected to reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, as illustrated in FIG. 7 or 9, the automated direct quantitative PCR system 1 provided herein comprises a liquid handling module 11 comprising a liquid dispenser 111 and a sample reader 112; a thermal cycling module 12 comprising a thermal cycler 121, a chip reader 122, one or more disposable PCR chips 123, a robotic arm 124, and a chip storage unit 125; an imaging module 13 comprising a light source 131 and a light detector 132; a control device 3; and reservoirs 301, 302, and 303; wherein the control device 3 is communicably connected to the liquid dispenser 111, sample reader 112, thermal cycler 121, chip reader 122, robotic arm 124, chip storage unit 125, light source 131, and light detector 132; and wherein the liquid dispenser 111 is fluidly connected to the reservoirs 301, 302, and 303. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In one embodiment, the control device 3 comprises a processor 21, a data processing module 22, and user interface 23, wherein the processor 21 is communicably connected to the data processing module 22 and user interface 23.

Direct Quantitative PCR Device

In one embodiment, provided herein is an automated direct quantitative PCR device 2, comprising a liquid handling module 11, a thermal cycling module 12, and an imaging module 13. In one embodiment, the liquid handling module 11 comprises a liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In another embodiment, provided herein is an automated direct quantitative PCR device 2, comprising a liquid handling module 11 comprising a liquid dispenser 111, a thermal cycling module 12, and an imaging module 13; wherein the liquid dispenser 111 comprises, in one embodiment, one or more fluid mixers 1111, in another embodiment, one or more fluid splitters 1112; and in yet another embodiment, one or more fluid mixers 1111 and one or more fluid splitters 1112. In certain embodiments, the liquid dispenser 111 further comprises a heating unit 1120.

In one embodiment, the automated direct quantitative PCR device 2 provided herein comprises a liquid handling module 11, a thermal cycling module 12, an imaging module 13, a processor 21, a data processing module 22, and a user interface 23; wherein the processor 21 is communicably connected to the liquid handling module 11, thermal cycling module 12, imaging module 13, data processing module 22, and user interface 23. In one embodiment, the liquid handling module 11 comprises a liquid dispenser 111, wherein the processor 21 is communicably connected to the liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In another embodiment, the automated direct quantitative PCR device 2 provided herein comprises a liquid handling module 11 comprising a liquid dispenser 111, a thermal cycling module 12, an imaging module 13, a processor 21, a data processing module 22, and a user interface 23; wherein the processor 21 is communicably connected to the liquid dispenser 111, thermal cycling module 12, imaging module 13, data processing module 22, and user interface 23; and wherein the liquid dispenser 111 is configured to be fluidly connected to one or more reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR device 2 provided herein comprises a liquid handling module 11 comprising a liquid dispenser 111 and a sample reader 112, a thermal cycling module 12, an imaging module 13, a processor 21, a data processing module 22, and a user interface 23; wherein the processor 21 is communicably connected to the liquid dispenser 111, sample reader 112, thermal cycling module 12, imaging module 13, data processing module 22, and user interface 23; and wherein the liquid dispenser 111 is fluidly connected to reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR device 2 provided herein comprises a liquid handling module 11; a thermal cycling module 12 comprising a thermal cycler 121, a chip reader 122, and one or more disposable PCR chips 123; an imaging module 13; a processor 21; a data processing module 22; and a user interface 23; wherein the processor 21 is communicably connected to the liquid handling module 11, thermal cycler 121, chip reader 122, imaging module 13, data processing module 22, and user interface 23. In one embodiment, the liquid handling module 11 comprises a liquid dispenser 111, wherein the processor 21 is communicably connected to the liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR device 2 provided herein comprises a liquid handling module 11; a thermal cycling module 12 comprising a thermal cycler 121, a chip reader 122, one or more disposable PCR chips 123, a robotic arm 124, and a chip storage unit 125; an imaging module 13; a processor 21; a data processing module 22; and a user interface 23; wherein the processor 21 is communicably connected to the liquid handling module 11, thermal cycler 121, chip reader 122, robotic arm 124, chip storage unit 125, imaging module 13, data processing module 22, and user interface 23. In one embodiment, the liquid handling module 11 comprises a liquid dispenser 111, wherein the processor 21 is communicably connected to the liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR device 2 provided herein comprises a liquid handling module 11, a thermal cycling module 12, an imaging module 13 comprising a light source 131 and a light detector 132, a processor 21, a data processing module 22, and a user interface 23; wherein the processor 21 is communicably connected to the liquid handling module 11, thermal cycling module 12, light source 131, light detector 132, data processing module 22, and user interface 23. In one embodiment, the liquid handling module 11 comprises a liquid dispenser 111, wherein the processor 21 is communicably connected to the liquid dispenser 111. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR device 2 provided herein comprises a liquid handling module 11 comprising a liquid dispenser 111; a thermal cycling module 12 comprising a thermal cycler 121, a chip reader 122, and one or more disposable PCR chips 123; an imaging module 13 comprising a light source 131 and a light detector 132; a processor 21; a data processing module 22; and a user interface 23; wherein the processor 21 is communicably connected to the liquid dispenser 111, thermal cycler 121, chip reader 122, light source 131, light detector 132, data processing module 22, and user interface 23; and wherein the liquid dispenser 111 is configured to be fluidly connected to one or more reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, as illustrated in FIG. 6, the automated direct quantitative PCR device 2 provided herein comprises a liquid handling module 11 comprising a liquid dispenser 111 and a sample reader 112; a thermal cycling module 12 comprising a thermal cycler 121, a chip reader 122, and one or more disposable PCR chips 123; an imaging module 13 comprising a light source 131 and a light detector 132; a processor 21; a data processing module 22; and a user interface 23; wherein the processor 21 is communicably connected to the liquid dispenser 111, sample reader 112, thermal cycler 121, chip reader 122, light source 131, light detector 132, data processing module 22, and user interface 23; and wherein the liquid dispenser 111 is configured to be fluidly connected to reservoirs 301, 302, and 303. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR device 2 provided herein comprises a liquid handling module 11 comprising a liquid dispenser 111; a thermal cycling module 12 comprising a thermal cycler 121, a chip reader 122, and one or more disposable PCR chips 123; and an imaging module 13 comprising a light source 131 and a light detector 132; wherein the liquid dispenser 111, thermal cycler 121, chip reader 122, light source 131, and light detector 132 are configured to be communicably connected to a control device 3; and wherein the liquid dispenser 111 is configured to be fluidly connected to one or more reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, as illustrated in FIG. 8, the automated direct quantitative PCR device 2 provided herein comprises a liquid handling module 11 comprising a liquid dispenser 111 and a sample reader 112; a thermal cycling module 12 comprising a thermal cycler 121, a chip reader 122, and one or more disposable PCR chips 123; and an imaging module 13 comprising a light source 131 and a light detector 132; wherein the liquid dispenser 111, thermal cycler 121, chip reader 122, light source 131, and light detector 132 are configured to be communicably connected to a control device 3; and wherein the liquid dispenser 111 is configured to be fluidly connected to reservoirs 301, 302, and 303. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR device 2 provided herein comprises a liquid handling module 11 comprising a liquid dispenser 111; a thermal cycling module 12 comprising a thermal cycler 121, a chip reader 122, one or more disposable PCR chips 123, a robotic arm 124, and a chip storage unit 125; an imaging module 13 comprising a light source 131 and a light detector 132; a processor 21; a data processing module 22; and a user interface 23; wherein the processor 21 is communicably connected to the liquid dispenser 111, thermal cycler 121, chip reader 122, robotic arm 124, chip storage unit 125, light source 131, light detector 132, data processing module 22, and user interface 23; and wherein the liquid dispenser 111 is configured to be fluidly connected to one or more reservoirs 30. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, as illustrated in FIG. 7, the automated direct quantitative PCR device 2 provided herein comprises a liquid handling module 11 comprising a liquid dispenser 111 and a sample reader 112; a thermal cycling module 12 comprising a thermal cycler 121, a chip reader 122, one or more disposable PCR chips 123, a robotic arm 124, and a chip storage unit 125; an imaging module 13 comprising a light source 131 and a light detector 132; a processor 21; a data processing module 22; and a user interface 23; wherein the processor 21 is communicably connected to the liquid dispenser 111, sample reader 112, thermal cycler 121, chip reader 122, robotic arm 124, chip storage unit 125, light source 131, light detector 132, data processing module 22, and user interface 23; and wherein the liquid dispenser 111 is configured to be fluidly connected to reservoirs 301, 302, and 303. In one embodiment, the liquid dispenser 111 comprises one or more fluid mixers and one or more fluid splitters. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In yet another embodiment, the automated direct quantitative PCR device 2 provided herein comprises a liquid handling module 11 comprising a liquid dispenser 111; a thermal cycling module 12 comprising a thermal cycler 121, a chip reader 122, one or more disposable PCR chips 123, a robotic arm 124, and a chip storage unit 125; and an imaging module 13 comprising a light source 131 and a light detector 132; wherein the liquid dispenser 111, thermal cycler 121, chip reader 122, robotic arm 124, chip storage unit 125, light source 131, and light detector 132 are configured to be communicably connected to a control device 3; and wherein the liquid dispenser 111 is configured to be fluidly connected to one or more reservoirs 30. In one embodiment, the liquid dispenser 111 comprises one or more mixers 1111 and one or more fluid splitters 1112. In certain embodiments, the liquid dispenser 111 further comprises a heating unit 1120.

In yet another embodiment, as illustrated in FIG. 9, the automated direct quantitative PCR device 2 provided herein comprises a liquid handling module 11 comprising a liquid dispenser 111 and a sample reader 112; a thermal cycling module 12 comprising a thermal cycler 121, a chip reader 122, one or more disposable PCR chips 123, a robotic arm 124, and a chip storage unit 125; and an imaging module 13 comprising a light source 131 and a light detector 132; wherein the liquid dispenser 111, sample reader 112, thermal cycler 121, chip reader 122, robotic arm 124, chip storage unit 125, light source 131, and light detector 132 are configured to be communicably connected to a control device 3; and wherein the liquid dispenser 111 is configured to be fluidly connected to reservoirs 301, 302, and 303. In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In one embodiment, the control device 3 comprises a processor 21, a data processing module 22, and user interface 23, wherein the processor 21 is communicably connected to the data processing module 22 and user interface 23.

In one embodiment, the automated direct quantitative PCR device 2 provided herein further comprises a communication module. In another embodiment, the automated direct quantitative PCR device 2 provided herein further comprises a UV light configured to decontaminate the space within the device.

In one embodiment, the automated direct quantitative PCR device 2 provided herein is portable. In another embodiment, the automated direct quantitative PCR device 2 provided herein is a point-of-care device, which can be used by a physician in a community clinic, an emergency room, an inpatient hospital, or an academic center. Thus, a physician can employ such a device to perform a number of diagnostic tests on site, thus eliminating the need to send the biological sample offsite. In yet another embodiment, the automated direct quantitative PCR device 2 provided herein is stationary. In yet another embodiment, the automated direct quantitative PCR device 2 provided herein is configured to run on a laboratory benchtop. In still another embodiment, the automated direct quantitative PCR device 2 provided herein is configured to provide a clinical diagnostic result within about 20 minutes, about 30, about 40 minutes.

In certain embodiments, the automated direct quantitative PCR device 2 provided herein is configured to analyze a fluid sample. In one embodiment, the fluid sample is a biological fluid sample. In another embodiment, the fluid sample is a biological fluid sample that may contain a nucleic acid. In yet another embodiment, the fluid sample is a biological fluid sample that may contain cells. In still another embodiment, the fluid sample is blood, cytosol, interstitial fluid, cytosol, plasma, saliva, serum, or saliva.

In certain embodiments, the fluid sample provided for analysis is in a container with machine-readable indicia, designating the sample information, including, but not limited to, sample source, sample type, and/or test to be performed. In one embodiment, the machine-readable indicia are a barcode or radio frequency identification (RFID) tag. In another embodiment, the machine-readable indicia are a barcode. In yet another embodiment, the machine-readable indicia is a one-dimensional or two-dimensional barcode. In still another embodiment, the machine-readable indicia are a RFID tag.

In one embodiment, the processor 21 is configured to receive data about the biological fluid sample to be analyzed, e.g., from a sample reader 112 and/or a user interface 23 as illustrated in FIGS. 1 to 9. In one embodiment, the sample reader 111 is a barcode reader, an optical character reader, or an RFID scanner (radio frequency tag reader).

Liquid Handling Module

In one embodiment, the liquid handling module 11 comprises a liquid dispenser 111. In one embodiment, the liquid dispenser 111 is a contact dispenser. In another embodiment, the liquid dispenser 111 is a non-contact dispenser. In yet another embodiment, the liquid dispenser 111 is a microdispenser. In yet another embodiment, the liquid dispenser 111 is a contact microdispenser. In still another embodiment, the liquid dispenser 111 is a non-contact microdispenser. In one embodiment, the liquid dispenser 111 is a fixed volume dispenser. In another embodiment, the liquid dispenser 111 is a variable volume dispenser.

In one embodiment, the liquid dispenser 111 is a piezoelectric dispenser. In another embodiment, the liquid dispenser 111 is an acoustic dispenser. In yet another embodiment, the liquid dispenser 111 is an inkjet dispenser. In yet another embodiment, the liquid dispenser 111 is a syringe-based dispenser. In still another embodiment, the liquid dispenser 111 is a solenoid-based dispenser.

In one embodiment, the liquid dispenser 111 is configured to accurately and reliably dispense a metered volume ranging from about 100 nL to about 100 μL, from about 500 nL to about 50 μL, from about 500 nL to about 20 μL, from about 500 nL to about 10μL, or from about 500 nL to about 5 μL. In another embodiment, the liquid dispenser 111 is configured to accurately and reliably dispense a metered volume ranging from about 500 nL to about 10 μL. In yet another embodiment, the liquid dispenser 111 is configured to accurately and reliably dispense a metered volume at about 1, about 2, about 3, about 4, about 5, about 6, about 7, about 8, about 9, or about 10 μL. In yet another embodiment, the liquid dispenser 111 is configured to accurately and reliably dispense a metered volume ranging from about 500 nL to about 5 μL. In still another embodiment, the liquid dispenser 111 is configured to accurately and reliably dispense a metered volume at about 1, about 2, about 3, about 4, or about 5 μL.

In certain embodiments, the liquid dispenser 111 is configured to aspirate a biological fluid sample into the liquid dispenser 111 and to dispense the biological fluid sample in a metered volume evenly into wells 1231 of a disposable PCR chip 123 in the thermal cycle module 12. In certain embodiments, the liquid dispenser 111 is configured to aspirate a predetermined volume of a biological fluid sample into the liquid dispenser 111, ranging from about 1 μL to about 1 mL, from about 2 μL to about 500 μL from about 10 μL to about 500 μL, from about 20 μL to about 250 μL, or from about 25 μL to about 100 μL. In certain embodiments, the liquid dispenser 111 is configured to aspirate a predetermined volume of a biological fluid sample into the liquid dispenser 111, ranging from about 25 μL to about 100 μL.

In one embodiment, the liquid dispenser 111 comprises a nozzle. In another embodiment, the liquid dispenser 111 comprises a plurality of nozzles. In yet another embodiment, the liquid dispenser 111 comprises a number of nozzles, ranging from about 1 to about 1,000, from about 1 to about 400, from about 1 to about 100, or from about 8 to about 64. In yet another embodiment, the liquid dispenser 111 comprises about 1, about 6, about 8, about 24, about 64, about 96, about 384, or about 1536 nozzles. In still another embodiment, the liquid dispenser 111 comprises about 1, about 8, or about 64 nozzles.

In one embodiment, liquid dispenser 111 is configured to be movable in relation to the housing 14 of the diagnostic device 1. In another embodiment, liquid dispenser 111 is configured to move to align nozzles with wells 1231 of a disposable PCR chip 123 in the thermal cycle module 12. In one embodiment, the liquid dispenser 111 is configured to move along the X-axis, Y-axis, and/or Z-axis in relation to the housing 14 of the diagnostic device 1. In another embodiment, the liquid dispenser 111 is configured to move along the X-axis and Y-axis in relation to the housing 14 of the diagnostic device 1. In yet another embodiment, the liquid dispenser 111 is configured to move along the X-axis and Z-axis in relation to the housing 14 of the diagnostic device 1.

In one embodiment, the liquid dispenser 111 comprises one or more fluid mixers 1111. In another embodiment, the liquid dispenser 111 comprises one or more fluid splitters 1112. In yet another embodiment, the liquid dispenser 111 comprises one or more fluid mixers 1111 and one or more fluid splitters 1112. In certain embodiments, the liquid dispenser 111 further comprises a heating unit 1120.

In certain embodiments, the liquid dispenser 111 comprises a number of fluid mixers 1111, ranging from about 1 to about 20, from about 1 to about 10, or from about 1 to about 5. In certain embodiments, the liquid dispenser 111 comprises 1, 2, 3, 4, or 5 fluid mixers 1111. In certain embodiments, the liquid dispenser 111 comprises one fluid mixer 1111. In certain embodiments, the liquid dispenser 111 comprises two fluid mixers 1111. In certain embodiments, the liquid dispenser 111 comprises three fluid mixers 1111. In certain embodiments, the one or more fluid mixers 1111 are serially connected. In certain embodiments, the one or more fluid mixers 1111 are in fluid communication.

In one embodiment, a fluid mixer 1111 is a static mixer. In another embodiment, a fluid mixer 1111 is an inline fluid mixer. In yet another embodiment, a fluid mixer 1111 comprises two or more inlet ports and one outlet port. In yet another embodiment, a fluid mixer 1111 comprises two or three inlet ports and one outlet port. In yet another embodiment, a fluid mixer 1111 comprises two inlet ports and one outlet port. In still another embodiment, a fluid mixer 1111 comprises three inlet ports and one outlet port.

In one embodiment, the liquid dispenser 111 comprises a fluid mixer 1111 having two inlet ports and one outlet port. In another embodiment, the liquid dispenser 111 comprises a fluid mixer 1111 having three inlet ports and one outlet port. In yet another embodiment, the liquid dispenser 111 comprises one fluid mixer 1111 having two inlet ports and one outlet port, and one fluid mixer 1111 having three inlet ports and one outlet port. In yet another embodiment, the liquid dispenser 111 comprises three fluid mixers 1111, each having two inlet ports and one outlet port.

In certain embodiments, the one or more fluid mixers 1111 are three serially connected fluid mixers, each having two inlet ports and one outlet port. In one embodiment, the first fluid mixer 1111 is configured to receive and mix together at least a portion of a biological fluid sample and a first buffer solution, in one embodiment, a neutralization buffer solution, from a reservoir 301 to form a first sample mixture. In another embodiment, the second fluid mixer 1113 is configured to receive and mix together at least a portion of the first sample mixture and a second buffer solution, in one embodiment, a dilution buffer solution (e.g., PBS), from a reservoir 302 to form a second sample mixture. In yet another embodiment, the third fluid mixer 1115 is configured to receive and mix together the second sample mixture and a third buffer solution, in one embodiment, a buffer solution suitable for a nucleic acid amplification reaction (e.g., PCR), from a reservoir 303 to form a third sample mixture ready to be dispensed in a metered volume evenly into wells 1231 of a disposable PCR chip 123 in the thermal cycle module 12. In one another embodiment, the third buffer solution comprises a dye. In another embodiment, the third buffer solution comprises a fluorescence dye. In yet another embodiment, the third buffer solution comprises a DNA-binding fluorescence dye. In one embodiment, the third buffer solution comprises a DNA polymerase and deoxyribonucleosides in a buffer solution suitable for a nucleic acid amplification (e.g., PCR) reaction. In another embodiment, the third buffer solution comprises a DNA polymerase, deoxyribonucleosides, and a DNA-binding fluorescence dye in a buffer solution suitable for a nucleic acid amplification (e.g., PCR) reaction.

In one embodiment, the fluid splitter is a static splitter. In another embodiment, the fluid splitter comprises one inlet ports and two outlet ports. In one embodiment, the fluid splitter is a splitter with a fixed split ratio, ranging from about 2:1 to about 100:1 or from about 5:1 to about 100:1. In another embodiment, the fluid splitter is a splitter with an adjustable split ratio ranging from about 2:1 to about 100:1, from about 5:1 to about 100:1, or from about 2:1 to about 100:1.

As illustrated in FIGS. 6 and 8, in one embodiment, the liquid dispenser 111 is enclosed within a housing 14. In one embodiment, the liquid dispenser 111 is configured to be in communication with a processor 21. In another embodiment, the liquid dispenser 111 is configured to be controlled by the processor 21. In yet another embodiment, the liquid dispenser 111 is configured to be controlled by a chip reader 122 in a thermal cycling module 12.

In one embodiment, the liquid handling module 11 further comprises a sample reader 112. In certain embodiments, the sample reader 112 is configured to identify an individual sample as it enters the system by reading a unique sample identification associated with the sample. In certain embodiments, the sample reader 112 is configured to read a machine-readable indicia from the container containing the biological fluid sample to be analyzed. In one embodiment, the sample reader 112 is a barcode reader. In another embodiment, the sample reader 112 is a RFID reader.

In certain embodiments, the sample reader 112 is configured to transmit the machine-readable indicia identifying indicia of the biological fluid sample to the processor 21. In certain embodiments, the sample reader 112 is configured to be accessible outside the housing 14.

As illustrated in FIGS. 6 to 9, in one embodiment, the liquid handling module 11 comprises a liquid dispenser 111, a sample reader 112, and a plurality of reservoirs 301, 302, and 303, where the liquid handler 111 is fluidly connected to the reservoirs 301, 302, and 303.

In certain embodiments, the liquid dispenser 111 is configured to dispense a metered volume of a sample to be analyze into wells 1231 of a disposable PCR chip 123 in a thermal cycle module 12.

In certain embodiments, the liquid dispenser 111 is configured to dispense a metered volume of a PCR reaction solution into wells 1231 of a disposable PCR chip 123 in a thermal cycle module 12. In certain embodiments, the PCR reaction solution is stored in a reservoir.

In certain embodiments, the liquid dispenser 111 is configured to dispense a metered volume of a liquid sealer to the wells 1231 of a disposable PCR chip 123 in a thermal cycle module 12 to minimize evaporation during thermal cycling. In certain embodiments, the liquid sealer is mineral oil or paraffin oil. In certain embodiments, the liquid sealer is stored in a reservoir.

In one embodiment, provided herein is an automated multiple-channel liquid dispenser 111, comprising a manifold; one or more pumps; one or more dispense heads; one or more fluid mixers 1111; and one or more fluid splitters 1112; wherein the manifold, pumps, dispense heads, fluid mixers 1111, and fluid splitters 1112 are in fluid communication.

In one embodiment, the liquid dispenser 111 further comprises a heating unit 1120 to heat a sample mixture. In certain embodiments, the heating unit 1120 is configure to heat a sample mixture to a temperature ranging from about 30 to about 100° C., from about 40 to about 90° C., or from about 50 to about 80° C. In certain embodiments, the heating unit 1120 is configure to heat a sample mixture to a temperature of about 40, about 45, about 50, about 55, about 60, about 65, about 70, about 75, or about 80° C. In certain embodiments, the heating unit 1120 is configure to heat a sample mixture for a period ranging from about 30 seconds to about 30 minutes, from about 1 to 20 minutes, or from about 2 to about 10 minutes. In certain embodiments, the heating unit 1120 is configure to heat a sample mixture for about 5, about 10, about 15, about 20, about 25, or about 30 minutes. In certain embodiments, the heating unit 1120 is configured to heat a sample mixture before the addition of a fluorescence dye. In certain embodiments, the heating unit 1120 is configured to heat a sample mixture from the first fluid mixer 1111. In certain embodiments, the heating unit 1120 is configured to heat a sample mixture from the second fluid mixer 1113. In certain embodiments, the heating unit 1120 is configured to heat a sample mixture from the third fluid mixer 1115. In certain embodiments, the heating unit 1120 is located between the first fluid mixter 1111 and the first fluid splitter 1112. In certain embodiments, the heating unit 1120 is located between the second fluid mixter 1113 and the second fluid splitter 1114. In certain embodiments, the heating unit 1120 is located between the second fluid mixter 1113 and the third fluid mixer 1115.

Thermal Cycler Module

As illustrated in FIG. 3, in one embodiment, the thermal cycling module 12 comprises a PCR thermal cycler 121, a chip reader 122, and one or more disposable PCR chips 123, where the PCR thermal cycler 121 and chip reader 122 are each independently configured to be in communication with a processor 21. In one embodiment, the PCR thermal cycler 121 is configured to be controlled by the processor 21. As illustrated in FIGS. 6 and 8, in one embodiment, the thermal cycling module 12 is enclosed within a housing 14.

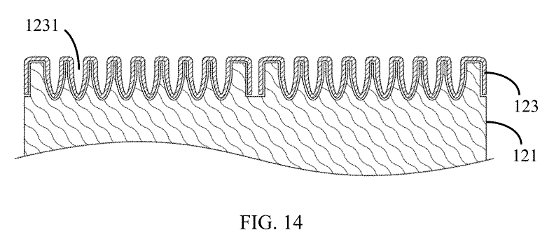

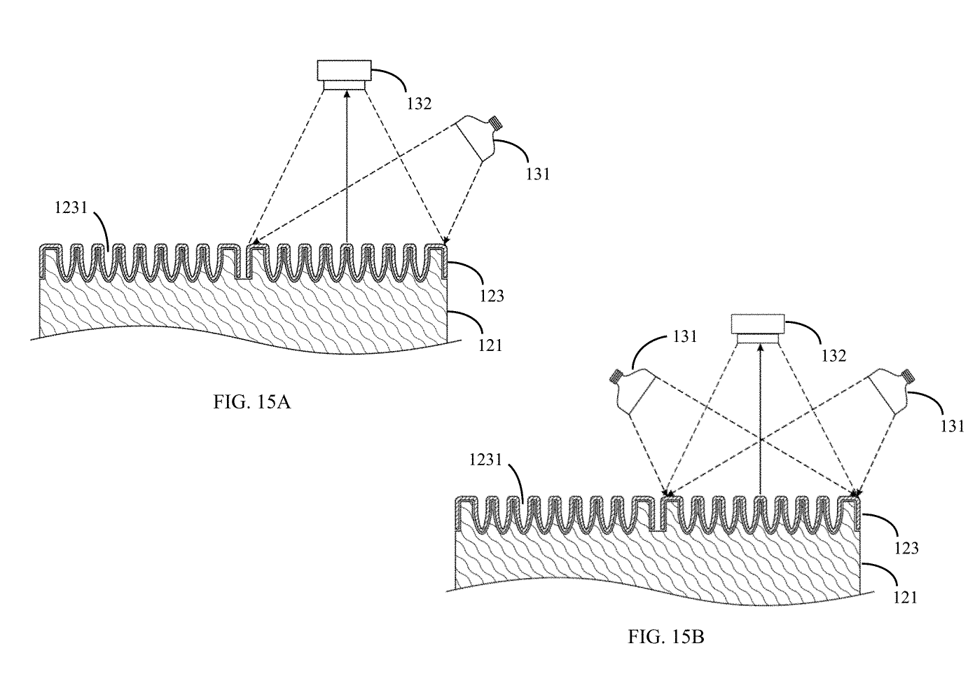

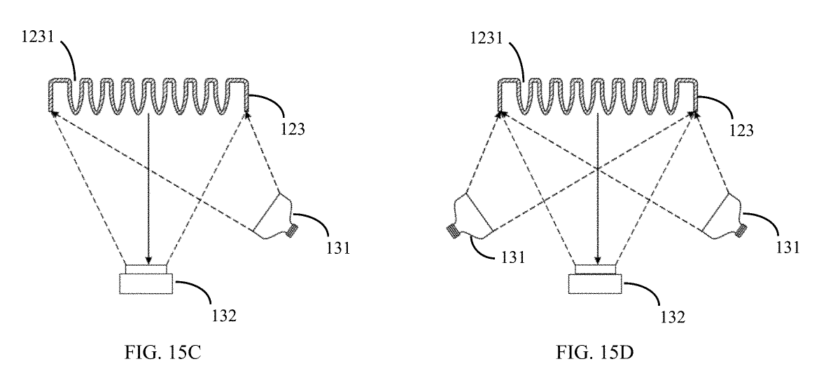

In one embodiment, the PCR thermal cycler 121 is a contact thermal cycler. In another embodiment, the PCR thermal cycler 121 is a contact thermal cycler having a close physical contact with the disposable PCR chips 123 for efficient thermal transfer. As illustrated in FIG. 14, in one embodiment, the top face of the PCR thermal cycler 121 has a contour closely matching the bottom contour of the disposable PCR chips 123 to maximize thermal transfer between the PCR thermal cycler 121 and the disposable PCR chips 123. In one embodiment, the PCR thermal cycler 121 is a metal plate having 256 wells, where the diameter of each well is 2.1 mm and the distance between each neighboring well is 4.5 mm.

In one embodiment, the PCR thermal cycler 121 is a metal block. In another embodiment, the PCR thermal cycler 121 is a block of aluminum, copper, silver, or a combination thereof. In yet another embodiment, the PCR thermal cycler 121 is an aluminum block. In yet another embodiment, the PCT thermal cycler 121 is a copper block. In yet another embodiment, the PCT thermal cycler 121 is a silver block. In still another embodiment, the PCR thermal cycler 121 is a Peltier device.

In one embodiment, the PCR thermal cycler 121 is a metal plate having a surface area ranging from about 10 cm² to about 1,000 cm², from about 25 cm² to about 500 cm², about 25 cm² to about 200 cm², or from about 25 cm² to about 100 cm². In another embodiment, the area is ranging from about 25 cm² to about 100 cm².

In another embodiment, the PCR thermal cycler 121 is a non-contact thermal cycler. In one embodiment, the PCR thermal cycler 121 is an air-based thermal cycler. In another embodiment, the PCR thermal cycler 121 is an infrared (IR) thermal cycler, a laser thermal cycler, an induction thermal cycler, or a microwave thermal cycler.

In one embodiment, the PCR thermal cycler 121 is configured to hold a plurality of disposable PCR chips 123 for thermal cycling. In another embodiment, the PCR thermal cycler 121 is configured to hold a number of disposable PCR chips 123 for thermal cycling, ranging from 1 to about 128, from 1 to about 64, from 1 to about 32, from 1 to about 16, or from 1 to about 4. In yet another embodiment, the PCR thermal cycler 121 is configured to hold from 1 to 4 disposable PCR chips 123 for thermal cycling.

In one embodiment, the PCR thermal cycler 121 is configured to operate at a temperature ranging from about 25 to about 100° C. or from about 50 to about 100° C. In another embodiment, the PCR thermal cycler 121 is configured to operate at a temperature ranging from about 50 to about 100° C. In yet another embodiment, the PCR thermal cycler 121 is configured to operate at about 55° C., about 72° C., and about 94° C.

In one embodiment, the PCR thermal cycler 121 is configured to perform a series of temperature changes that are repeated for a number of cycles, ranging from about 2 to about 100, from about 10 to about 50, or from about 10 to about 40 cycles. In another embodiment, the PCR thermal cycler 121 is configured to perform a series of temperature changes that are repeated for about 10 to about 40 cycles. In yet another embodiment, the PCR thermal cycler 121 is configured to perform a series of temperature changes that are repeated for about 10, about 15, about 20, about 25, about 30, about 35, or about 40 cycles. In still another embodiment, the PCR thermal cycler 121 is configured to perform a series of temperature changes that are repeated for about 30 cycles.

In one embodiment, in each cycle, the PCR thermal cycler 121 is configured to reach a temperature around 95° C. first to allow the double-stranded chain separation of a target DNA; subsequently to a temperature around 50-60° C. to allow the binding of primers with the target DNA template; and then to a temperature around 68-72° C. to facilitate the polymerization carried out by a DNA polymerase.

In one embodiment, the PCR thermal cycler 121 is configured to operate at a heating rate ranging from about 1 to about 100° C./s, from about 2 to about 50° C./s, or from about 2 to about 25° C./s. In another embodiment, the PCR thermal cycler 121 is configured to operate at a cooling rate ranging from about 1 to about 100° C./s, from about 2 to about 50° C./s, or from about 2 to about 25° C./s. In one embodiment, the PCR thermal cycler 121 is configured to complete PCR amplification within a period of time ranging from about 10 to about 60 minutes, from about 15 to 30 minutes, or from about 15 to 20 minutes. In another embodiment, the PCR thermal cycler 121 is configured to complete PCR amplification within about 15, about 20, about 25, or 30 minutes. In yet another embodiment, the PCR thermal cycler 121 is configured to complete 30 PCR cycles within a period of time ranging from about 10 to about 60 minutes, from about 15 to 30 minutes, or from about 15 to 20 minutes. In still another embodiment, the PCR thermal cycler 121 is configured to complete 30 PCR cycles within about 15, about 20, about 25, or 30 minutes.

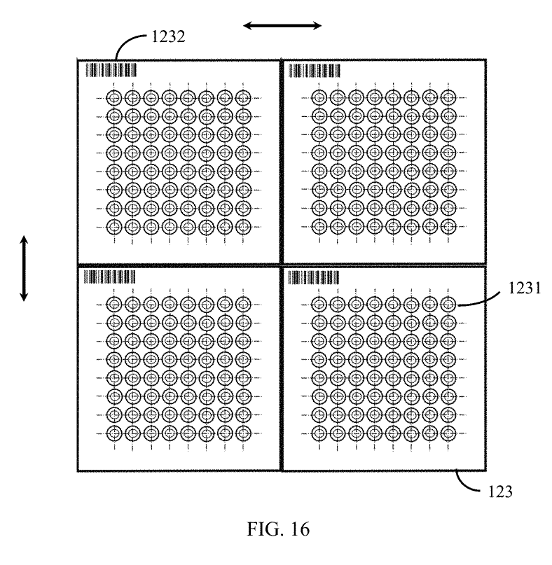

In one embodiment, the PCR thermal cycler 121 is configured to be movable in relation to the housing 14 of the diagnostic device 1. In one embodiment, the PCR thermal cycler 121 is configured to move along the X-axis in relation to the housing 14 of the diagnostic device 1. In another embodiment, the PCR thermal cycler 121 is configured to move along the Y-axis in relation to the housing 14 of the diagnostic device 1. As illustrated in FIG. 16, in one embodiment, the PCR thermal cycler 121 is configured to move along the X-axis and Y-axis in relation to the housing 14 of the diagnostic device 1.

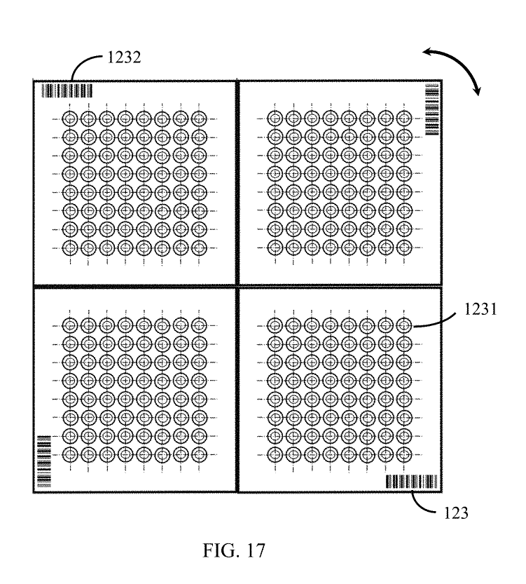

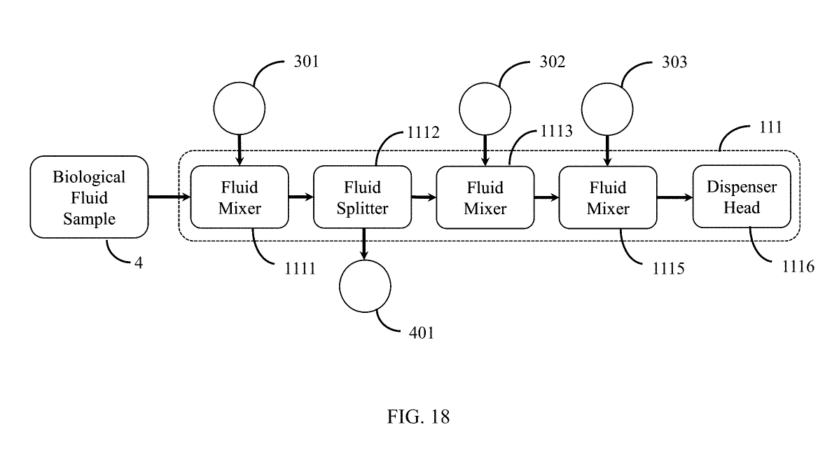

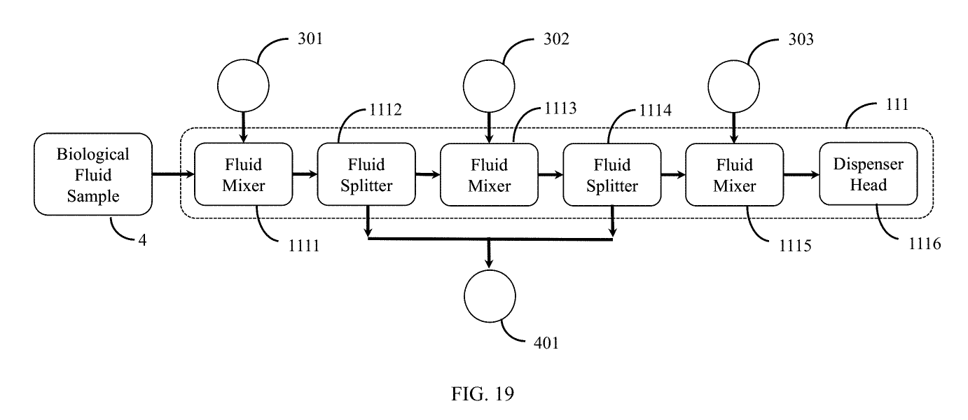

In one embodiment, the PCR thermal cycler 121 is configured to be rotatable in relation to the housing 14 of the diagnostic device 1. As illustrated in FIG. 17, in one embodiment, the PCR thermal cycler 121 is configured to rotate along the Z-axis in relation to the housing 14 of the diagnostic device 1. In one embodiment, the PCR thermal cycler 121 is configured to rotate in a step of 90 degree.

In one embodiment, the PCR thermal cycler 121 comprises a stepping motor.

As illustrated in FIGS. 6 and 8, in one embodiment, the PCR thermal cycler 121 is enclosed within a housing 14.

In one embodiment, the chip reader 122 is configured to read a machine-readable indicia 1232 from a disposable PCR chip 123. In one embodiment, the chip reader 122 is a barcode reader. In another embodiment, the chip reader 122 is a RFID reader.

As illustrated in FIG. 4, in another embodiment, the thermal cycling module 12 further comprises a robotic arm 124 and a PCR chip storage 125, where the robotic arm 124 and chip storage 125 are each independently configured to be in communication with a processor 21.

In one embodiment, the robotic arm 124 is a Cartesian robot arm, a Gantry robot arm, or a Select Compliant Articulated Robot arm (SCARA). In one embodiment, the robotic arm 124 is configured to transfer a disposable PCR chip 123 from the chip storage 125 into or onto the PCR thermal cycler 121. In another embodiment, the robotic arm 124 is configured to transfer a disposable PCR chip 123 from the chip storage 125 and to place the disposable PCR chip 123 onto the top surface of the contact thermal cycler 121 with the top contour of the thermal cycler 121 aligned with the bottom contour of the disposable PCR chips 123. In yet another embodiment, the robotic arm 123 is configured to remove the disposable PCR chip 123 from the PCR thermal cycler 121 for disposal.

In one embodiment, the robotic arm 124 is configured to move along the X-axis or Y-axis in relation to the housing 14 of the diagnostic device 1. In another embodiment, the robotic arm 124 is configured to move along the Z-axis in relation to the housing 14 of the diagnostic device 1. In yet another embodiment, the robotic arm 124 is configured to move along the X-axis and Z-axis in relation to the housing 14 of the diagnostic device 1. In still another embodiment, the robotic arm 124 is configured to move along the Y-axis and Z-axis in relation to the housing 14 of the diagnostic device 1.

In one embodiment, the chip storage 125 is configured to house a plurality of disposable PCR chips 123. In one embodiment, the chip storage 125 is configured to house a number of disposable PCR chips 123, ranging from about 4 to about 500, from about 4 to about 200, from about 4 to about 100, from about 4 to about 50, or from about 4 to about 20 disposable PCR chips 123. In certain embodiments, the chip storage 125 is a hotel, a carousel or a rack. In certain embodiments, the chip storage 125 is configured to be accessible by the robotic arm 124 to transfer disposable PCR chips 123, one at a time. In certain embodiments, the chip storage 125 is configured to be accessible by the robotic arm 124 within the housing 14 to transfer disposable PCR chips 123, one at a time.

In certain embodiments, the robotic arm 124 and chip storage 125 are each independently controlled by the processor 21. As illustrated in FIGS. 7 and 9, in one embodiment, the robotic arm 124 and chip storage 125 are enclosed within a housing 14. In certain embodiments, the housing 14 has an opening (e.g., a door) to provide access to the chip storage 125 from the outside of the housing 14.

In one embodiment, the disposable PCR chip 123 comprises a plurality of wells 1231, where each well 1231 functions as a reaction chamber. In another embodiment, the disposable PCR chip 123 comprises a number of wells 1231, ranging from about 10 to about 10,000, from about 20 to about 5,000, from about 50 to about 500, or from about 50 to about 100. In yet another embodiment, the disposable PCR chip 123 comprises from 50 to 100 wells 1231. In yet another embodiment, the disposable PCR chip 123 comprises 6, 24, 64, 96, 384, or 1536 wells 1231. In yet another embodiment, the disposable PCR chip 123 comprises 64 wells 1231. In yet another embodiment, the disposable PCR chip 123 comprises 8×8 wells 1231. In one embodiment, all the wells 1231 have the same volume for reaction. In another embodiment, each wells 1231 has a volume ranging from about 1 μL to about 1 mL or from about 10 μL to about 500 μL.

In one embodiment, the disposable PCR chip 123 further comprises a machine-readable indicia 1232. In one embodiment, the machine-readable indicia 1232 are a barcode or radio frequency identification (RFID) tag. In another embodiment, the machine-readable indicia 1231 are a barcode. In yet another embodiment, the machine-readable indicia 1231 is a one-dimensional or two-dimensional barcode. In still another embodiment, the machine-readable indicia 1231 are a RFID tag.

In one embodiment, the machine-readable indicia 1232 are configured to provide an identification for a disposable PCR chip 123. In another embodiment, the machine-readable indicia 1232 are configured to provide a unique identification for a disposable PCR chip 123. In certain embodiments, this identification number is linked to information about the disposable PCR chip 123, including, but not limited to, serial number, expiration date, predetermined biological assays or diagnostic tests to be performed, predetermined liquid dispensing parameters to be used, predetermined thermal cycling parameters to be used, predetermined imaging parameters to be used, and/or predetermined data processing parameters to be used.

In certain embodiments, the machine-readable indicia 1232 are configured to determine the alignment of a disposable PCR chip 123 on a contact thermal cycler 121. In certain embodiments, if misalignment is detected, the disposable PCR chip 123 is adjusted to be aligned using, for example, the robotic arm 124.

In one embodiment, the disposable PCR chip 123 is a plastic plate. In another embodiment, the disposable PCR chip 123 is a plastic plate fabricated from polydimethylsiloxane (PDMS), polymethylmethacrylate (PMMA), polycarbonate, polypropylene, cyclic olefin polymer (COP), or a mixture thereof. In yet another embodiment, the disposable PCR chip 123 is fabricated from silicon or glass.

In one embodiment, the disposable PCR chip 123 comprises wells 1221 that each comprises a pair of primers and a non-specific amplification blocker. In one embodiment, the non-specific amplification blocker is a peptide nucleic acid (PNA), a locked nucleic acid (LNA), a morpholino, a glycol nucleic acid (GNA), threose nucleic acid (TNA), a bridged nucleic acid (BNA), a N3′-P5′ phosphoramidate (NP) oligomer, a minor groove binder-linked-oligonucleotide (MGB-linked oligonucleotide), a phosphorothioate (PS) oligomer, a C₁₋₄ alkylphosphonate oligomer, a phosphoramidate, a β-phosphodiester oligonucleotide, an α-phosphodiester oligonucleotide, or a combination thereof. In another embodiment, the non-specific amplification blocker is a peptide nucleic acid (PNA), a locked nucleic acid (LNA), a morpholino, a glycol nucleic acid (GNA), threose nucleic acid (TNA), a bridged nucleic acid (BNA), a phosphorothioate (PS) oligomer, a C₁₋₄ alkylphosphonate oligomer, a phosphoramidate, a β-phosphodiester oligonucleotide, an α-phosphodiester oligonucleotide, or a combination thereof. In yet another embodiment, the non-specific amplification blocker is a peptide nucleic acid (PNA), a locked nucleic acid (LNA), a C₁₋₄ alkylphosphonate oligomer, or a combination thereof. In yet another embodiment, the non-specific amplification blocker is a peptide nucleic acid (PNA). In yet another embodiment, the non-specific amplification blocker is a C₁₋₄ alkylphosphonate oligomer. In still another embodiment, the non-specific amplification blocker is a methylphosphonate oligomer.

In one embodiment, the disposable PCR chip 123 is sealed during storage. In one embodiment, the thermal cycling module 12 further comprises a de-lidder configured to remove a lid or a seal from a disposable PCR chip 123. In another embodiment, the de-lidder is configured to place a lid onto a disposable PCR chip 123 before thermal cycling.

Imaging Module

As illustrated in FIGS. 5 to 9, in one embodiment, the imaging module 13 comprises a light source 131 and a light detector 132, where the light source 131 and light detector 132 are each independently configured to be in communication with a processor 21. In certain embodiments, the light source 131 and light detector 132 are each independently configured to be controlled by the processor 21. As illustrated in FIGS. 6 to 9, in one embodiment, the imaging module 13 is enclosed within a housing 14.

As illustrated in FIGS. 5 to 9, in one embodiment, the imaging module 13 comprises two light sources 131 and a light detector 132, where the light sources 131 and light detector 132 are each independently configured to be in communication with a processor 21. In certain embodiments, the light sources 131 and light detector 132 are each independently configured to be controlled by the processor 21.

In one embodiment, the imaging module 13 is configured to be stationary in relation to the housing 14 of the diagnostic device 1. In another certain embodiment, the imaging module 13 is configured to be movable in relation to the housing 14 of the diagnostic device 1. In one embodiment, the imaging module 13 is configured to be movable translationally in one, two, or three dimensions in relation to the housing 14 of the diagnostic device 1. In another embodiment, the imaging module 13 is configured to be rotatable in relation to the housing 14 of the diagnostic device 1.

In one embodiment, the light source 131 is configured to emit light in the absorption bands of one or more fluorescent dyes. In another embodiment, the light source 131 is configured to emit light in an absorption band of a fluorescent dye. In yet another embodiment, the light source 131 is configured to selectively emit light in the absorption bands of one or more fluorescent dyes. In still another embodiment, the light source 131 is configured to selectively emit light in an absorption band of a fluorescent dye.

In one embodiment, the fluorescent dye is a DNA intercalating dye. In another embodiment, the DNA intercalating dye is ethidium bromide, EVAGREEN®, a SYBR® dye, an oxazole yellow dye, a thiazole orange dye, a PICOGREEN® dye, a SYTO® dye, or a combination thereof. In certain embodiments, the DNA intercalating dye is EVAGREEN®. In certain embodiments, the DNA intercalating dye is a SYBR® dye. In certain embodiments, the DNA intercalating dye is an oxazole yellow dye. In certain embodiments, the DNA intercalating dye is a thiazole orange dye. In certain embodiments, the DNA intercalating dye is a PICOGREEN® dye. In one embodiment, the DNA intercalating dye is a SYTO® dye. In another embodiment, the DNA intercalating dye is a SYTO® blue dye. In yet another embodiment, the DNA intercalating dye is a SYTO® green dye. In yet another embodiment, the DNA intercalating dye is a SYTO® orange dye. In yet another embodiment, the DNA intercalating dye is SYTO® 80, SYTO® 81, SYTO® 82, SYTO® 83, SYTO® 84, or SYTO® 85. In still another embodiment, the DNA intercalating dye is a SYTO® red dye.

In certain embodiments, the light source 131 comprises one or more light filters configured to provide light comprising one or more specified wavelengths. In one embodiment, the one or more light filters comprise one or more dichroics. In another embodiment, the one or more light filters are configured to be in communication with a processor 21. In yet another embodiment, the one or more light filters are configured to be controlled by the processor 21. In certain embodiments, the light source 131 comprises a rotating disk with two to six optical filters to provide two to six specified wavelengths.

In one embodiment, the light source 131 is configured to emit light having a wavelength ranging from about 400 to about 700 nm, from about 450 to about 650 nm, or from about 500 to about 600 nm. In another embodiment, the light source 131 is configured to emit light having a wavelength ranging from about 500 to about 600 nm. In yet another embodiment, the light source 131 is configured to emit light having a wavelength of about 500, about 510, about 520, about 530, about 540, about 550, about 560, about 570, about 580, about 590, or about 600 nm. In still another embodiment, the light source 131 is configured to emit light having a wavelength of 540 nm.

In one embodiment, the light source 131 is configured to selectively emit light having a wavelength ranging from about 400 to about 700 nm, from about 450 to about 650 nm, or from about 500 to about 600 nm. In another embodiment, the light source 131 is configured to selectively emit light having a wavelength ranging from about 500 to about 600 nm. In yet another embodiment, the light source 131 is configured to selectively emit light having a wavelength of about 500, about 510, about 520, about 530, about 540, about 550, about 560, about 570, about 580, about 590, or about 600 nm. In still another embodiment, the light source 131 is configured to selectively emit light having a wavelength of 540 nm.

As illustrated in FIG. 14, in one embodiment, the light source 131 is configured to provide an excitation beam having an angle (α) from the top of the disposable PCR chip 123, wherein the angle is ranging from 0 to about 90 degrees. In one embodiment, the light source 131 is configured to provide an excitation beam that causes a substantially uniform excitation across all the reaction chambers 1231 in a disposable PCR chip 123. In another embodiment, the light source 131 is configured to provide an excitation beam that causes a substantially uniform excitation across the reaction chambers 1231 within an area of a disposable PCR chip 123, where the area is ranging from about 1 cm² to about 1,000 cm², from about 1 cm² to about 100 cm², about 10 cm² to about 50 cm², about 20 cm² to about 50 cm², or about 10 cm² to about 25 cm². In one embodiment, the area is ranging from about 10 cm² to about 25 cm².

In one embodiment, the light source 131 is configured to be stationary in relation to the housing 14 of the diagnostic device 1. In another embodiment, the light source 131 is configured to be movable in relation to the housing 14 of the diagnostic device 1. In one embodiment, the light source 131 is configured to be movable translationally in one, two, or three dimensions in relation to the housing 14 of the diagnostic device 1. In another embodiment, the light source 131 is configured to be rotatable in relation to the housing 14 of the diagnostic device 1. As illustrated in FIGS. 6 to 9, in one embodiment, the light source 131 is disposed within a housing 14.

In one embodiment, the light source 131 is a laser, a light emitting diode (LED), or a light bulb. In another embodiment, the light source 131 is a laser. In yet another embodiment, the light source 131 is a LED. In still another embodiment, the light source 131 is a light bulb. In certain embodiments, the light source 131 is a mercury arc lamp, a xenon arc lamp (XBO), or a metal halide lamp.

In one embodiment, the light detector 132 is configured to detect light in the emission bands of the one or more fluorescent dyes. In another embodiment, the light detector 132 is configured to detect light in an emission band of the fluorescent dye. In one embodiment, the light detector 132 is configured to selectively detect light in the emission bands of the one or more fluorescent dyes. In another embodiment, the light detector 132 is configured to selectively detect light in an emission band of the fluorescent dye.

In certain embodiments, the light detector 132 comprises one or more light filters to selectively detect light having one or more specified wavelengths. In one embodiment, the one or more light filters are one or more dichroics. In another embodiment, the one or more light filters are configured to be in communication with a processor 21. In yet another embodiment, the one or more light filters are configured to be controlled by the processor 21. In certain embodiments, the light detector 132 comprises a rotating disk with two to six optical filters to detect two to six specified wavelengths.

In one embodiment, the light detector 132 is configured to detect light having a wavelength ranging from about 400 to about 700 nm, from about 450 to about 650 nm, from about 500 to about 600 nm, or from about 550 to about 600 nm. In another embodiment, the light detector 132 is configured to detect light having a wavelength ranging from about 550 to about 600 nm. In yet another embodiment, the light detector 132 is configured to detect light having a wavelength of about 500, about 510, about 520, about 530, about 540, about 550, about 560, about 570, about 580, about 590, or about 600 nm. In still another embodiment, the light detector 132 is configured to detect light at 560 nm.

In one embodiment, the light detector 132 is configured to selectively detect light having a wavelength ranging from about 400 to about 700 nm, from about 450 to about 650 nm, from about 500 to about 600 nm, or from about 550 to about 600 nm. In another embodiment, the light detector 132 is configured to selectively detect light having a wavelength ranging from about 550 to about 600 nm. In yet another embodiment, the light detector 132 is configured to selectively detect light having a wavelength of about 500, about 510, about 520, about 530, about 540, about 550, about 560, about 570, about 580, about 590, or about 600 nm. In still another embodiment, the light detector 132 is configured to selectively detect light at 560 nm.

In one embodiment, the light detector 132 is a camera. In another embodiment, the light detector 132 is a charge-coupled device (CCD) camera. In one embodiment, the CCD camera has a pixel size ranging from about 1 to about 50 μm or from about 2 to about 30 μm. In another embodiment, the CCD camera has a pixel number ranging from about 1 to about 50 megapixels. Because of its wider field-of-view in comparison with spot detectors such as a photodiode and photomultiplier tube, a CCD camera is suitable for high throughput fluorescence imaging applications.

As illustrated in FIG. 14, in one embodiment, the light detector 132 is configured to be substantially perpendicular to the top face of the disposable PCR chip 123. In one embodiment, the light detector 132 is configured to simultaneously detect light emitted from all the reaction chambers 1231 in a disposable PCR chip 123. In another embodiment, the light detector 132 is configured to simultaneously detect light emitted from all the reaction chambers 1231 within an area of a disposable PCR chip 123, where the area is ranging from about 1 cm² to about 1,000 cm², from about 1 cm² to about 100 cm², about 10 cm² to about 50 cm², about 20 cm² to about 50 cm², or about 10 cm² to about 25 cm². In one embodiment, the area is ranging from about 10 cm² to about 25 cm².

In one embodiment, the light detector 132 is configured to be stationary in relation to the housing 14 of the diagnostic device 1. In another embodiment, the light detector 132 is configured to be movable in relation to the housing 14 of the diagnostic device 1. In one embodiment, the light detector 132 is configured to be movable translationally in one, two, or three dimensions in relation to the housing 14 of the diagnostic device 1. In another embodiment, the light detector 132 is configured to be rotatable in relation to the housing 14 of the diagnostic device 1. As illustrated in FIGS. 6 to 9, in one embodiment, the light detector 132 is disposed within a housing 14.

In one embodiment, the light source(s) 131 and the light detector 132 are configured for detection from the top face of a disposable PCR chip 123. In another embodiment, the light source(s) 131 and the light detector 132 are configured for detection from the bottom face of a disposable PCR chip 123.

In one embodiment, the imaging module 13 is configured to determine the quality of liquid dispensing by measuring the fluorescence intensity of each well of a disposable PCR chip after liquid dispension, but before amplification to determine whether the fluorescence intensity of each well falls within a predetermined range to ensure an accurate volume of a liquid is delivered into the well.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL, (microliters); mM (millimolar); μM (micromolar); hr or hrs (hours); min (minutes); PBS (phosphate-buffered saline).

For all of the following examples, standard procedures known to those skilled in the art can be utilized. Unless otherwise indicated, all temperatures are expressed in ° C. (degrees Centigrade). All procedures are conducted at room temperature unless otherwise noted. The procedures illustrated herein are intended to exemplify the applicable technology through the use of specific examples and are not indicative of the scope of the disclosure.

### Example 1

### Identification of CYP 2D6 Mutation

Cytochrome P450 (CYP) 2D6 is an enzyme involved in the metabolism of antibiotics in the body. A number of SNPs (single nucleotide polymorphism) are found in human CYP 2D6 genes. Certain SNPs cause higher activity of the P450 enzyme, which could accelerate the metabolism of antibiotics. Certain SNPs cause lower activity of the P450 enzyme, which could extend the half time of the antibiotics. CYP 2D6 A2C mutation was identified in less than 1 hour as described below.

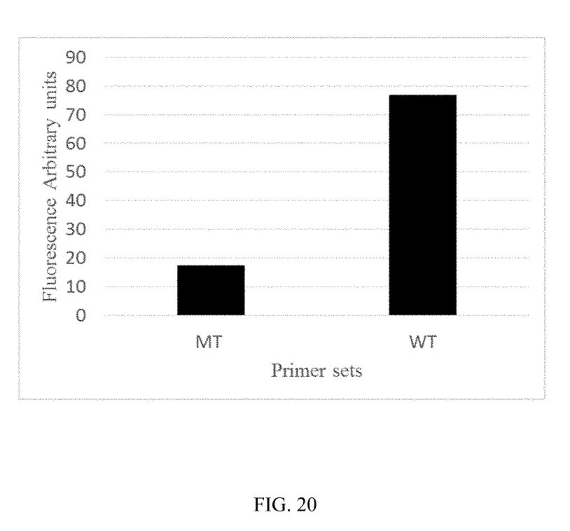

One drop of human blood (about 25 μL) in a sample collection tube preloaded with 100 μL of blood lysis buffer was vortexed for 30 seconds to lysis the cells. After addition of a neutralization buffer (100 μL), the sample solution was diluted 100 fold with PBS. The diluted sample (1 μL) was then mixed with 20 μL of a Taq DNA polymerase mix containing a fluorescence dye indicator. The mixture was subsequently aliquoted into wells of a disposable PCR chip, each well containing a pair of specific PCR primers for A2C mutation and non-specific target blocker. The PCR reaction mixtures in the wells of the PCR chip was thermally cycled for amplification. The fluorescence intensity was determined using a camera. The results are shown in FIG. 20.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A direct quantitative PCR device, comprising a liquid dispenser, a thermal cycler, a light source, and a light detector; wherein the liquid dispenser comprises one or more fluid splitters.

2. A direct quantitative PCR device comprising a liquid handling module comprising a liquid dispenser, a thermal cycling module; and an imaging module; wherein the liquid dispenser comprises one or more fluid splitters.

3. The device of claim 1, wherein the liquid dispenser is a non-contact liquid dispenser.

4. The device of claim 1, wherein the liquid dispenser is configured to dispense a metered volume ranging from about 500 nL to about 10 μL.

5. (canceled)

6. The device of claim 1, wherein the liquid dispenser further comprises one or more fluid mixers.

7-20. (canceled)

21. The device of claim 1, wherein the liquid dispenser comprises one fluid splitter.

22-25. (canceled)

26. The device of claim 1, wherein the liquid dispenser comprises two fluid splitters.

27-32. (canceled)

33. The device of claim 1, wherein the liquid dispenser is configured to dispense a metered volume ranging from about 500 nL to about 10 μL.

34. The device of claim 2, wherein the thermal cycling module comprises a thermal cycler.

35. The device of claim 1, wherein the thermal cycler is a direct contact thermal cycler.

36. The device of claim 1, wherein the thermal cycler is a metal block.

37. The device of claim 1, wherein the thermal cycler is configured to operate at a heating rate ranging from about 1 to about 100° C./s.

38. The device of claim 1, wherein the thermal cycler is configured to operate at a cooling rate ranging from about 1 to about 100° C./s.

39. The device of claim 2, wherein the imaging module comprises a light source and a light detector.

40. The device of claim 1, wherein the light source is configured to produce an excitation beam that causes a substantially uniform excitation across within an area ranging from about 25 cm² to about 100 cm².

41. The device of claim 1, wherein the light source is a LED light.

42. The device of claim 1, wherein the light detector is a camera.

43. (canceled)

44. The device of claim 1, wherein the light source and light detector are configured to determine the quality of liquid dispensing by the liquid dispenser.

45. The device of claim 1, wherein the derive is integrated into a single housing.

46. The device of claim 1, wherein the device is a point-of-care device.

47. A direct quantitative PCR method, comprising:
(a) mixing a biological fluid sample to be analyzed with one or more buffer solutions with a liquid dispenser comprising one or more fluid mixers and one or more fluid splitters to form a sample mixture; wherein one of the one or more buffer solutions comprises a fluorescence dye;
(b) dispensing a predetermined volume of the sample mixture into each well of a disposable PCR chip;
(c) determining the fluorescence intensity of the fluorescence dye in each well of the disposable PCR chip;
(d) subjecting the sample mixture in each well of the disposable PCR chip to suitable nucleic acid amplification conditions; and
(e) determining the fluorescence intensity of the fluorescence dye in each well of the disposable PCR chip after amplification.

48-79. (canceled)
